(19) United States
(12) Patent Application Publication
(10) Pub. No.: US 2009/0220488 A1
(43) Pub. Date: Sep. 3, 2009

(54) EVALUATING AND TREATING SCLERODERMA

(76) Inventor: Humphrey Gardner, Salem, MA (US)

Correspondence Address: BIOGEN IDEC / FINNEGAN HENDERSON, LLP, 901 NEW YORK AVENUE, NW, WASHINGTON, DC, 20001-4413, US

(21) Appl. No.: 12/065,217

(22) PCT Filed: Aug. 24, 2006
(86) PCT No.: PCT/US06/33125

§ 371 Date: Aug. 5, 2008

Related U.S. Application Data
(60) Provisional application No. 60/712,998, filed on Aug. 31, 2005.

(51) Int. Cl. A61K 39/395; A61K 38/02; A61K 31/7088; A61K 31/7105; C12Q 1/02; C12Q 1/68

(52) U.S. Cl. 424/130.1; 514/2; 514/44 R; 514/44 A; 435/29; 435/6

(57) ABSTRACT

The expression of a number of genes is altered in scleroderma. Therapeutic methods for treating scleroderma can include counteracting the effects of the altered gene expression profile. Further, scleroderma can be diagnosed and monitored by evaluating the expression of one or more of the altered genes.

### CROSS-REFERENCE TO RELATED APPLICATIONS

This application claims priority to U.S. Application Ser. No. 60/712,998, filed on Aug. 31, 2005, the contents of which are hereby incorporated by reference in their entirety.

### SUMMARY

Scleroderma is a condition manifested by the appearance of a hard type skin. The condition includes a group of connective tissue and rheumatic disorders, including localized scleroderma (morphea and linear scleroderma) and systemic scleroderma (limited scleroderma, diffuse scleroderma, and sine scleroderma).

The expression of a number of genes is altered in scleroderma. Therapeutic methods for treating scleroderma can include counteracting the effects of the altered gene expression profile. Further, scleroderma can be diagnosed and monitored by evaluating the expression of one or more of the altered genes.

In one aspect, this disclosure features a method of treating or preventing scleroderma or an other fibrotic disorder. The method includes administering, to a subject, e.g., a human subject, a Wnt signalling antagonist, e.g., in an amount effective to treat or prevent scleroderma. The subject is typically a human, e.g., a human who has or who is at risk for scleroderma or other fibrotic disorder. The subject can be a human who has been identified as having decreased WIF1 expression in a skin biopsy.

A Wnt signalling antagonist is an agent that decreases Wnt signalling in a cell of the subject. The antagonist can inhibit a canonical Wnt, e.g., Wnt1, Wnt3A, or Wnt8, and/or a non-canonical Wnt, e.g., Wnt4, Wnt5A, or Wnt11. The antagonist can be a protein or a small molecule. For example, the antagonist can be an agent that inhibits interaction between a Wnt and a cell surface receptor for Wnt, e.g., a canonical Wnt and Frizzled or LRP5/6.

In one embodiment, the Wnt signalling antagonist is a Wnt binding protein, e.g., an antibody that binds to Wnt or a protein at least 90, 95, 97, 98, or 99% identical, or identical to, to a naturally occurring Wnt binding protein or a functional fragment thereof, e.g., Wnt binding fragment. Examples of a naturally occurring Wnt binding protein, e.g., an sFRP protein (soluble frizzled-related protein), WIF-1, or Cerberus.

In one embodiment, the Wnt signalling antagonist is a protein that binds to a cell surface receptor for Wnt. For example, the protein can be an antibody that binds to a cell surface receptor for Wnt, e.g., a Frizzled protein or an LRP, e.g., LRP5/6. In particular, the antibody can bind to an extracellular region of the cell surface receptor for Wnt. The protein can be at least 90, 95, 97, 98, or 99% identical, or identical to, to a naturally occurring protein that interacts with a cell surface receptor for Wnt. For example, the protein is at least 90, 95, 97, 98, or 99% identical, or identical to, a functional fragment of a Dickkopf protein, e.g., Dkk-1, Dkk-2, Dkk-3, or Dkk-4.

A Wnt signalling antagonist can be a protein (e.g., an artificial transcription factor that represses transcription) or nucleic acid agent (e.g., siRNA, anti-sense, or aptamer) that decreases expression (or activity) of a positively acting component of the Wnt pathway, e.g., a Wnt protein or a Wnt receptor. Alternatively, a Wnt signalling antagonist can be a protein (e.g., an artificial transcription factor that activates transcription) or nucleic acid agent (e.g., gene therapy vector) that increases expression of a negatively acting component of the Wnt pathway, e.g., a Wnt inhibitor such as an artificial or naturally occurring Wnt inhibitor (e.g., an sFRP, WIF, or Cerberus protein).

In another aspect, the disclosure features a method of treating or preventing scleroderma. The method includes administering, to a subject, an agent that increases activity or expression of WIF (e.g., WIF1) or an sFRP protein, e.g., in an amount effective to treat or prevent scleroderma. The agent can be, for example, a protein that includes a functional fragment of a WIF or sFRP protein or a nucleic acid that encodes such a protein. In one embodiment, the agent is a Wnt-binding fragment of WIF1. In another embodiment, the agent includes a full-length, mature WIF1.

In another aspect, the disclosure features a method of treating or preventing scleroderma. The method includes administering, to a subject, an IGF binding agent or an inhibitor of an IGF, in an amount effective to treat or prevent scleroderma. For example, the IGF binding agent binds to IGF I or IGF II. The IGF binding agent can include an IGF binding region of a naturally occurring IGFBP, e.g., IGFBP-3. In one embodiment, the IGF binding agent includes a full length, mature IGFBP. In one embodiment, the IGF binding agent includes an antibody that binds to IGF I or IGF II.

In another aspect, the disclosure features a method of treating or preventing scleroderma. The method includes: administering, to a subject, an agent that (i) increases expression of a gene encoding gene product in columns 2, 4, or 6 of Table 1, or (ii) increases activity of a gene product encoded by the gene. The agent can be administered in an amount effective to treat or prevent scleroderma. In one embodiment, the agent is a nucleic acid that includes a sequence that encodes a protein that includes a functional fragment of the gene product. For example, the nucleic acid is in a viral vector and delivered using viral particle. In one embodiment, the agent is a protein, e.g., a protein that includes a functional fragment of the gene product. For example, the agent includes the gene product itself.

In still another aspect, the disclosure features a method of treating or preventing scleroderma. The method includes administering, to a subject, an agent that (i) decreases expression of a gene encoding a gene product in columns 1, 3, or 5 of Table 1, or (ii) decreases activity of a gene product encoded by the gene. The agent can be administered in an amount effective to treat or prevent scleroderma. In one embodiment, the agent is a nucleic acid antagonist of gene expression, e.g., an RNAi, e.g., an siRNA. In another embodiment, the agent is an antibody that binds to the gene product.

In another aspect, the disclosure features a method of evaluating a subject. The method includes: obtaining a sample (e.g., a skin biopsy or serum sample) from a subject; and evaluating expression of a gene in Table 1 in cells in the biopsy. An alteration in expression of the gene relative to a reference is indicative of scleroderma or risk for scleroderma. With respect to a gene listed in columns 2, 4, and 6 of Table 1, a decrease in expression of the gene relative to a reference is indicative of scleroderma or risk for scleroderma; whereas with respect to a gene listed in columns 1, 3, and 5 of Table 1, an increase in expression of the gene relative to a reference is indicative of scleroderma or risk for scleroderma. Samples can be used without culturing and passaging of cells within the sample.

In one embodiment, the gene encodes WIF1. A decrease in WIF 1 expression relative to a reference is indicative of scleroderma or risk for scleroderma.

The evaluating can include a quantitative or qualitative evaluation of expression levels. For example, the reference is a parameter obtained by evaluating a normal subject who does not have scleroderma.

In one embodiment, a plurality of genes is evaluated. The expression of each of the genes can be compared to corresponding references, e.g. values (quantitative or qualitative values) for the expression of same genes based on a reference sample or for a statistical assessment, e.g., an average of a cohort of matched subjected, e.g., a cohort of subjects who have scleroderma or a cohort of subject who do not have scleroderma, e.g., healthy subjects. Information from evaluating the plurality of genes can be used to obtain a profile of gene expression. The profile can be compared to a corresponding reference profile, e.g., a profile based on a reference sample or for a statistical assessment, e.g., an average of a cohort of matched subjected, e.g., a cohort of subjects who have scleroderma or a cohort of subject who do not have scleroderma, e.g., healthy subjects. Profiles can be compared, e.g., using a distance function.

In another aspect, the disclosure features a method of evaluating a subject. The method includes: obtaining a sample (e.g., a skin biopsy, serum sample, or other sample) from a subject; and evaluating expression of a collagen in cells in the sample, wherein a increase in collagen expression relative to a reference is indicative of scleroderma or risk for scleroderma. Collagen expression can be evaluated by detecting mRNA encoding collagen or by detecting collagen protein (including fragments thereof). The method can include administering a therapy for scleroderma to the subject, if the subject is indicated for scleroderma or risk for scleroderma. For example, the therapy is a therapy described herein. In one embodiment, the collagen is collagen XI. The method can include detecting fragments of the collagen, e.g., collagen XI fragments (e.g., N and C propeptides). Fragments can be detected, e.g., using an antibody. The method can further include preparing a report indicating a diagnosis of scleroderma or risk for scleroderma using results of the evaluating.

In another aspect, the disclosure features a computer-readable database that includes a plurality of records. Each of which includes: a) a first field that comprises information about skin pathology of a subject; and b) a second field that comprises information about expression of a gene in Table 1 in cells from a sample (e.g., a skin biopsy or serum sample) obtained from the subject. For example, each record of the plurality further includes a field that comprises information identifying the subject. Each record of the plurality can further include additional fields that include information about expression of a gene in Table 1 such that each record includes information for a plurality of genes in Table 1, e.g., at least 2, 4, 5, 7, 8, 9, or 10 genes from one or more columns in Table 1, e.g., at least 5, 10, 20, or 25% of the genes in one or more columns of Table 1.

### DETAILED DESCRIPTION

Agents that inhibit Wnt signalling or increase IGFBP activity can be used to treat scleroderma or another fibrotic disorder, as can agents that alter the expression or activity of the proteins listed in Table 1. Changes in gene expression that accompany scleroderma also provide a reference for identifying other therapeutic agents and for diagnostic methods of evaluating subjects.

### Wnt Signalling

Decreased expression of a Wnt inhibitor protein, WIF1, is characteristic of scleroderma biopsies. Accordingly, therapies that decrease Wnt pathway signalling can be used to treat or prevent scleroderma and other fibrotic disorders. Wnt proteins are secreted glycoproteins that mediate important cell signalling functions. Wnts include canonical Wnts (e.g., Wnt1, Wnt3a, and Wnt8). These Wnts can signal by stabilizing β-catenin and activating transcription mediated by Tcf/LEF. Wnts also include noncanonical Wnts, such as Wnt4, Wnt5A, and Wnt11. The noncanonical Wnts can activate alternative signalling pathways include Ca²⁺ signals. Many Wnt signals are transduced by cell surface receptors, e.g., receptors of the Frizzled (Fr) family and low-density lipoprotein receptor-related proteins (LRP), particularly LRP5 and LRP6.

A number of naturally occurring proteins function as inhibitors of Wnt signalling. These proteins include proteins that bind directly to Wnt, such as members of the sFRP class of inhibitors, e.g., the sFRP family itself, WIF-1, and Cerberus. Other naturally occurring proteins that function as inhibitors include the Dickkopf class which includes Dkk-1 through Dkk-4. These proteins interact with LRP5/6 to inhibit Wnt signalling.

### IGFBPs and the IGF Pathway

Decreased expression of insulin-like growth factor binding protein-3 (IGFBP-3) is characteristic of scleroderma biopsies. Accordingly, therapies that decrease IGF activity can be used to treat or prevent scleroderma and other fibrotic disorders. Insulin-like growth factor binding proteins (IGFBPs) are secreted proteins that bind to and sequester insulin-like growth factor (IGF), e.g., IGF-1 or IGF-2. IGFs are secreted growth factors that can act as potent mitogens that activate cell proliferation and differentiation. IGFBPs have high affinity for IGFs, e.g., better than 10⁻¹⁰ M.

In addition to using IGFBP-3 as a therapeutic agent, it is possible to use antibodies or other agents that bind to and inhibit an IGF, e.g., IGF-1 or IGF-2.

### Functionally Related Proteins

The use of a particular protein described herein can also be implemented using a related, but different protein that provides the same function, e.g., a protein that includes a functional fragment of that particular protein and that is related by sequence homology to the protein, e.g., at least 90, 95, 97, 98, or 99% identical to the particular protein within the region of the functional fragment, or at least 90, 95, 97, 98, or 99% with respect to the full-length of the particular protein (referring usually to the mature version of secreted and/or processed proteins). The protein can differ, e.g., by at least one, 2, 3, 4, 5, or 8 amino acids, and, e.g., by fewer than 25, 20, 15, 12, 10, 8, 7, 6, 4, 3 residues.

Calculations of sequence identity between two sequences are performed by aligning the sequences for optimal comparison purposes (e.g., gaps can be introduced in one or both of a first and a second amino acid or nucleic acid sequence for optimal alignment and non-homologous sequences can be disregarded for comparison purposes) and then counting the number of identical positions shared by the sequences, taking into account the number of gaps, and the length of each gap, which need to be introduced for optimal alignment of the two sequences. The determination of sequence identity is typically calculated using the GAP program in the GCG software package, using a Blossum 62 scoring matrix with a gap penalty of 12, a gap extend penalty of 4, and a frameshift gap penalty of 5.

Related proteins may also be encoded by nucleic acids that hybridize to one another, e.g., under medium stringency, high stringency, or very high stringency conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either can be used. Specific hybridization conditions include: i) medium stringency hybridization conditions in 6×SSC at about 45° C., followed by one or more washes in 0.2×SSC, 0.1% SDS at 60° C.; ii) high stringency hybridization conditions in 6×SSC at about 45° C., followed by one or more washes in 0.2×SSC, 0.1% SDS at 65° C.; and iii) very high stringency hybridization conditions are 0.5M sodium phosphate, 7% SDS at 65° C., followed by one or more washes at 0.2×SSC, 1% SDS at 65° C.

Further a useful protein may have one or more mutations (e.g., deletions, insertions, or substitutions) relative to a particular protein described herein (e.g., a conservative or non-essential amino acid substitutions), which do not have a substantial effect on function. Whether or not a particular substitution will be tolerated, can be predicted, e.g., by aligning closely related natural proteins to identify conserved and non-conserved positions, by mutagenesis experiments (e.g., alanine scanning), by inspecting structural models, or by consulting tables of related residues, e.g., as described in Bowie, et al. (1990) Science 247:1306-1310. Generally, a conservative amino acid substitution is one in which the amino acid residue is replaced with an amino acid residue having a similar side chain. Families of amino acid residues having similar side chains include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Functionally related proteins can be identified by a variety of methods. For example, a nucleic acid encoding a protein can be subjected to mutagenesis and then evaluated in a functional assay for the protein, e.g., using cultured cells.

Useful methods for mutagenesis include PCR mutagenesis, saturation mutagenesis, cassette mutagenesis, alanine scanning, and oligonucleotide directed mutagenesis. A library of random amino acid sequence variants can also be generated by the synthesis of a set of degenerate oligonucleotide sequences. PCR mutagenesis can be performed by reducing the fidelity of Taq polymerase so that random mutations are introduced during replication, e.g., by using a dGTP/dATP ratio of five and adding Mn²⁺ to the PCR reaction. (Leung et al., 1989, Technique 1:11-15). The pool of amplified DNA fragments can be inserted into appropriate cloning vectors to provide random mutant libraries. Saturation mutagenesis allows for the rapid introduction of a large number of single base substitutions into cloned DNA fragments (Mayers et al., 1985, Science 229:242). This technique includes generation of mutations, e.g., by chemical treatment or irradiation of single-stranded DNA in vitro, and synthesis of a complimentary DNA strand. A library of variants can also be generated from a set of degenerate oligonucleotide sequences. Chemical synthesis of degenerate sequences can be carried out in an automatic DNA synthesizer, and the synthetic genes then ligated into an appropriate expression vector.

Non-random or directed, mutagenesis techniques can be used to provide specific sequences or mutations in specific regions. These techniques can be used to create variants which include, e.g., deletions, insertions, or substitutions, of residues in the amino acid sequence of the protein. The sites for mutation can be modified individually or in series, e.g., by (1) substituting first with conserved amino acids and then with more radical choices depending upon results achieved, (2) deleting the target residue, or (3) inserting residues of the same or a different class adjacent to the located site, or combinations of these options. Oligonucleotide-mediated mutagenesis is a useful method for preparing substitution, deletion, and insertion variants of DNA. See, e.g., Adelman et al., (DNA 2:183, 1983). Alanine scanning mutagenesis is a useful method for identification of certain residues or regions of the desired protein that are preferred locations or domains for mutagenesis, Cunningham and Wells (Science 244:1081-1085, 1989). Cassette mutagenesis can also be used, e.g., as described in Wells et al. (1985) Gene, 34:315, and can be used to create, e.g., combinatorial libraries of variants.

### Screening Methods

Test compounds can be screened to identify compounds useful for the prevention or treatment of scleroderma or other fibrotic disorder. A test compound can be any chemical compound, for example, a macromolecule (e.g., a polypeptide, a protein complex, or a nucleic acid) or a small molecule (e.g., an amino acid, a nucleotide, an organic or inorganic compound). The test compound can have a formula weight of less than about 10,000 grams per mole, less than 5,000 grams per mole, less than 1,000 grams per mole, or less than about 500 grams per mole. The test compound can be naturally occurring (e.g., a herb or a nature product), synthetic, or both. Examples of macromolecules are proteins, protein complexes, and glycoproteins, nucleic acids, e.g., DNA, RNA and PNA (peptide nucleic acid). Examples of small molecules are peptides, peptidomimetics (e.g., peptoids), amino acids, amino acid analogs, polynucleotides, polynucleotide analogs, nucleotides, nucleotide analogs, organic or inorganic compounds e.g., heteroorganic or organometallic compounds. A test compound can be the only substance assayed by the method described herein. Alternatively, a collection of test compounds can be assayed either consecutively or concurrently by the methods described herein. Exemplary test compounds can be obtained from a combinatorial chemical library including peptide libraries (see, e.g., U.S. Pat. No. 5,010,175, Furka, Int. J. Pept. Prot. Res. 37:487-493 (1991) and Houghton et al., Nature 354:84-88 (1991)), peptoids (e.g., WO 91/19735), encoded peptides (e.g., WO 93/20242), random bio-oligomers (e.g., WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, et al. infra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. No. 5,525,735 and U.S. Pat. No. 5,519,134; morpholino compounds, U.S. Pat. No. 5,506,337; benzodiazepines, U.S. Pat. No. 5,288,514, and the like). Libraries of aptamers can also be evaluated.

The test compound or compounds can be screened individually or in parallel. A compound can be screened by being monitored the level of expression of one or more genes encoding a protein in Table 1. Comparing a compound-associated expression profile to a reference profile can identify the ability of the compound to modulate gene expression in dermal tissue, e.g., to alter fibroblast behavior or otherwise treat or prevent a fibrotic disorder, such as scleroderma. The expression profile can be a profile based on one or more genes mentioned herein, e.g., a gene encoding a protein listed in Table 1. An example of the parallel screening is a high throughput drug screen. A high-throughput method can be used to screen large libraries of chemicals. Such libraries of test compounds can be generated or purchased e.g., from Chembridge Corp., San Diego, Calif. Libraries can be designed to cover a diverse range of compounds. For example, a library can include 10,000, 50,000, or 100,000 or more unique compounds. Alternatively, prior experimentation and anecdotal evidence, can suggest a class or category of compounds of enhanced potential. A library can be designed and synthesized to cover such a class of chemicals. A library can be tested on cell lines, such as scleroderma fibroblasts, and gene expression levels can be monitored. Regardless of a method used for screening, compounds that alter the expression level are considered “candidate” compounds or drugs. Candidate compounds are retested on cells from scleroderma samples, or tested on animals. Candidate compounds that are positive in a retest are considered “lead” compounds.

Once a lead compound has been identified, standard principles of medicinal chemistry can be used to produce derivatives of the compound. Derivatives can be screened for improved pharmacological properties, for example, efficacy, pharmacokinetics, stability, solubility, and clearance. The moieties responsible for a compound's activity in the assays described above can be delineated by examination of structure-activity relationships (SAR) as is commonly practiced in the art. A person of ordinary skill in pharmaceutical chemistry could modify moieties on a lead compound and measure the effects of the modification on the efficacy of the compound to thereby produce derivatives with increased potency. For an example, see Nagarajan et al. (1988) J. Antibiot. 41: 1430-8. Furthermore, if the biochemical target of the lead compound is known or determined, the structure of the target and the lead compound can inform the design and optimization of derivatives. Molecular modeling software is commercially available (e.g., Molecular Simulations, Inc.).

### Expression Monitoring

Agents for treating scleroderma and other disorders described herein can be identified by selecting agents that modulate expression of a gene described herein, e.g., a gene encoding a protein in Table 1, e.g., WIF1, an IGFBP, or collagen XI. Any method can be used to evaluate an agent for its ability to modulate gene expression. For example, a cell is contacted with a candidate compound and mRNA or protein expression is evaluated, e.g., relative to the level of expression in the absence of the candidate compound. When expression is greater in the presence of the candidate compound than in its absence, the candidate compound is identified as a stimulator of a gene expression. Alternatively, when expression is less (e.g., statistically significantly less) in the presence of the candidate compound than in its absence, the candidate compound is identified as an inhibitor of gene expression.

Methods for detecting gene expression in a sample include detecting mRNA, detecting cDNA, or detecting protein, e.g., using an antibody or other binding protein, or using an activity assay. Exemplary molecular techniques include RT-PCR and microarray analysis. Many of these techniques can be used to obtain qualitative or quantitative values for gene expression. For example, QT-PCR can be used to provide a quantitative value for expression of a gene of interest. These techniques can be used to evaluate one or more genes encoding a gene product listed in Table 1, e.g., WIF-1, an IGFBP, or collagen XI.

Examples of methods of gene expression analysis include DNA arrays or microarrays (Brazma and Vilo, FEBS Lett., 2000, 480, 17-24; Celis, et al., FEBS Lett., 2000, 480, 2-16), SAGE (serial analysis of gene expression) (Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. US A., 2000, 97, 1976-81), protein arrays and proteomics (Celis, et al., supra; Jungblut, et al., Electrophoresis, 1999, 20, 2100-10), expressed sequence tag (EST) sequencing (Celis, et al., supra; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (e.g., Jurecic and Belmont, Curr. Opin. Microbiol., 2000, 3, 316-21), and mass spectrometry methods (reviewed in To, Comb. Chem. High Throughput Screen, 2000, 3, 235-41).

Reporter genes can be used to evaluate changes in gene expression. Exemplary regulatory sequences include those located within 100, 200, 500, 700, or 1600 basepairs of the mRNA start site of the gene of interest, e.g., WIF1, IGFBP3, or a gene in Table 1.

Reporter genes can be made by operably linking a regulatory sequence to a sequence encoding a reporter gene. A number of methods are available for designing reporter genes. For example, the sequence encoding the reporter protein can be linked in frame to all or part of the sequence that is normally regulated by the regulatory sequence. Such constructs can be referred to as translational fusions. It is also possible to link the sequence encoding the reporter protein to only regulatory sequences, e.g., the 5′ untranslated region, TATA box, and/or sequences upstream of the mRNA start site. Such constructs can be referred to as transcriptional fusions. Still other reporter genes can be constructed by inserting one or more copies (e.g., a multimer of three, four, or six copies) of a regulatory sequence into a neutral or characterized promoter.

Reporter genes can be introduced into germline cells of non-human mammals, e.g., to produce transgenic animals. Reporter genes can also be introduced into culture cells, e.g., tissue culture cells, e.g., fibroblasts.

Exemplary reporter proteins include chloramphenicol acetyltransferase, green fluorescent protein and other fluorescent proteins (e.g., artificial variants of GFP), beta-lactamase, beta-galactosidase, luciferase, and so forth. The reporter protein can be any protein other than the protein encoded by the endogenous gene that is subject to analysis. Epitope tags can also be used. The reporter protein is preferably stable and rapidly degraded.

Exemplary methods can include evaluating a transgene that includes a reporter gene for the gene of interest of a transgenic mammal for altered expression of a reporter gene (e.g., a GFP or variant protein). The transgenic mammal can be administered a test compound, and, if the compound modulates expression of the reporter gene, the test compound is selected.

Similarly, compounds can be screened using a cell-based assay, e.g., using cultures of cells that contain a reporter whose expression is operably linked to a regulatory sequence from the gene of interest (e.g., from a promoter, enhancer, untranslated region, upstream or downstream of the coding sequence).

### Antibodies

Antibodies can be used to modulate activity of a Wnt signalling pathway. Particularly useful antibodies bind to a secreted component of a Wnt signalling pathway or an extracellular region of a component of a Wnt signalling pathway. An antibody can be selected based on whether it antagonizes or agonize Wnt signalling.

For example, one class of antibodies includes molecules that bind to Wnt and inhibit Wnt activity, e.g., inhibit Wnt binding to a cell surface receptor, e.g., a frizzled receptor or LRP5/6. Another class of antibodies includes molecules that bind to the extracellular region of a cell surface receptor for Wnt, such as a frizzled receptor or LPR5/6, and reduce or prevent Wnt interaction with the receptor or otherwise reduce receptor signalling.

The term “antibody” refers to a protein comprising at least one immunoglobulin variable domains. A typical antibodies includes a heavy chain variable domain and a light chain variable domains, but a camelid antibody may have only a single variable immunoglobulin domain. Immunoglobulin variable domains include into regions of hypervariability, termed “complementarity determining regions” (“CDR”), interspersed with regions that are more conserved, termed “framework regions” (FR). The extent of the framework region and CDRs has been precisely defined (see, Kabat, E. A., et al. (1991) Sequences of Proteins of Immunological Interest, Fifth Edition, US Department of Health and Human Services, NIH Publication No. 91-3242, and Chothia, C. et al. (1987) J. Mol. Biol. 196:901-917). Each variable domain is composed of three CDRs and four FRs, arranged from amino-terminus to carboxy-terminus in the following order: FR1, CDR1, FR2, CDR2, FR3, CDR3, FR4.

The antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH₁, CH₂ and CH₃. The light chain constant region is comprised of one domain, CL. The variable domain of the heavy and light chains contains a binding domain that interacts with an antigen. The constant regions of the antibodies typically mediate the binding of the antibody to host tissues or factors, including various cells of the immune system and the first component (C1q) of the classical complement system.

As used herein, the term “immunoglobulin” refers to a protein that includes one or more polypeptides that have a domain that forms an immunoglobulin fold. An immunoglobulin domain is roughly a cylinder (about 4×2.5×2.5 nm) with two extended protein layers: one layer contains three strands of polypeptide chain and the other contains four. In each layer the adjacent strands are antiparallel and form a β-sheet. The two layers are roughly parallel and are often connected by a single intrachain disulfide bond. An immunoglobulin can include a region encoded by an immunoglobulin gene. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin genes and gene segments.

The term “antigen-binding fragment” of an antibody (or simply “antibody portion,” or “fragment”), as used herein, refers to one or more fragments of a full-length antibody that retain the ability to specifically bind to an antigen. Examples of binding fragments encompassed within the term “antigen-binding fragment” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) one or more complementarity determining regions (CDR) that retain antigen-binding ability, in the absence of a complete variable domain. Furthermore, although the two domains of the Fv fragment, VL and VH, are coded for by separate genes, they can be joined, using recombinant methods, by a synthetic linker that enables them to be made as a single protein chain in which the VL and VH domains pair to form monovalent molecules (known as single chain Fv (scFv); see e.g., Bird et al. (1988) Science 242:423-426; and Huston et al. (1988) Proc. Natl. Acad. Sci. USA 85:5879-5883) that are antigen-binding fragments of an antibody.

An “effectively human” immunoglobulin variable domain is an immunoglobulin variable domain that includes a sufficient number of human framework amino acid positions such that the immunoglobulin variable domain does not elicit an immunogenic response in a normal human. An “effectively human” antibody is an antibody that includes a sufficient number of human amino acid positions such that the antibody does not elicit an immunogenic response in a normal human. Human and effectively human immunoglobulin variable domains and antibodies can be used as therapeutics for human subjects.

Antibodies can be made by immunizing an animal (e.g., non-human animals and non-human animals include human immunoglobulin genes) with the relevant antigen or a fragment thereof. Such antibodies may be obtained using the entire mature protein as an immunogen, or by using fragments (e.g., soluble fragments and small peptides). The peptide immunogens additionally may contain a cysteine residue at the carboxyl terminus, and are conjugated to a hapten such as keyhole limpet hemocyanin (KLH). Additional peptide immunogens may be generated by replacing tyrosine residues with sulfated tyrosine residues. Methods for synthesizing such peptides include, for example, as in Merrifield, J. Amer. Chem. Soc. 85, 2149-2154 (1963); Krstenansky, et al., FEBS Lett. 211, 10 (1987). Antibodies can also be made by selecting antibodies from a protein expression library, e.g., a phage display library.

Human monoclonal antibodies (mAbs) directed against target proteins can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., WO 91/00906, WO 91/10741; WO 92/03918; WO 92/03917; Lonberg et al. 1994 Nature 368:856-859; Green. et al. 1994 Nature Genet. 7:13-21; Morrison et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

Monoclonal antibodies can also be generated by other methods including methods that use recombinant DNA technology. An alternative method, referred to as the “combinatorial antibody display” method, has been developed to identify and isolate antibody fragments having a particular antigen specificity, and can be utilized to produce monoclonal antibodies (for descriptions of combinatorial antibody display see e.g., Sastry et al. 1989 PNAS 86:5728; Huse et al. 1989 Science 246:1275; and Orlandi et al. 1989 PNAS 86:3833). After immunizing an animal with an immunogen as described above, the antibody repertoire of the resulting B-cell pool is cloned. The DNA sequence of the variable domains of a diverse population of antibodies can be obtained using a mixture of oligomer primers and PCR. For instance, mixed oligonucleotide primers corresponding to the 5′ leader (signal peptide) sequences and/or framework 1 (FR1) sequences, as well as primer to a conserved 3′ constant region primer can be used for PCR amplification of the heavy and light chain variable domains from a number of murine antibodies (Larrick et al., 1991, Biotechniques 11:152-156). A similar strategy can also been used to amplify human heavy and light chain variable domains from human antibodies (Larrick et al., 1991, Methods: Companion to Methods in Enzymology 2:106-110).

Chimeric antibodies, including chimeric immunoglobulin chains, can be produced by recombinant DNA techniques. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see PCT/US86/02269; EP 184,187; EP 171,496; EP 173,494; WO 86/01533; U.S. Pat. No. 4,816,567; EP 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl. Cancer Inst. 80:1553-1559).

An antibody or an immunoglobulin chain can be humanized. Humanized antibodies, including humanized immunoglobulin chains, can be generated by replacing sequences of the Fv variable domain which are not directly involved in antigen binding with equivalent sequences from human Fv variable domains. Humanized antibodies can be produced by a variety of methods, including CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539. Still other methods for generating humanized antibodies are provided by Morrison, S. L., 1985, Science 229:1202-1207, by Oi et al., 1986, BioTechniques 4:214, and U.S. Pat. No. 5,585,089, U.S. Pat. No. 5,693,761 and U.S. Pat. No. 5,693,762.

In some implementations, monoclonal, chimeric and humanized antibodies can be modified by, e.g., deleting, adding, or substituting other portions of the antibody, e.g., the constant region. For example, an antibody can be modified as follows: (i) by deleting the constant region; (ii) by replacing the constant region with another constant region, e.g., a constant region meant to increase half-life, stability or affinity of the antibody, or a constant region from another species or antibody class; or (iii) by modifying one or more amino acids in the constant region to alter, for example, the number of glycosylation sites, agonist cell function, Fc receptor (FcR) binding, complement fixation, among others.

Antibody constant regions can be altered. Antibodies with altered function, e.g. altered affinity for an agonist ligand, such as FcR on a cell, or the C1 component of complement can be produced by replacing at least one amino acid residue in the constant portion of the antibody with a different residue (see e.g., EP 388,151 A1, U.S. Pat. No. 5,624,821 and U.S. Pat. No. 5,648,260). Similar type of alterations could be described which if applied to the murine, or other species immunoglobulin would reduce or eliminate these functions.

To identify an antibody that not only binds, but also has a particular function (e.g., inhibits), an antibody can be evaluated in a functional assay. For example, a plurality of antibodies that bind to a target (e.g., Wnt or a Wnt receptor) can be evaluated in this manner.

### Nucleic Acid Antagonists

In certain implementations, nucleic acid antagonists are used to decrease expression of a target protein, e.g., a positively acting component of the Wnt pathway (e.g., Wnt or a Wnt receptor), an IGF protein, or other protein whose expression is upregulated in tissue from scleroderma patients, e.g., a protein listed in Table 2. In one embodiment, the nucleic acid antagonist is an siRNA that targets mRNA encoding the target protein. Other types of antagonistic nucleic acids can also be used, e.g., a nucleic acid aptamer, a dsRNA, a ribozyme, a triple-helix former, or an antisense nucleic acid.

siRNAs are small double stranded RNAs (dsRNAs) that optionally include overhangs. For example, the duplex region of an siRNA is about 18 to 25 nucleotides in length, e.g., about 19, 20, 21, 22, 23, or 24 nucleotides in length. Typically the siRNA sequences are exactly complementary to the target mRNA. dsRNAs and siRNAs in particular can be used to silence gene expression in mammalian cells (e.g., human cells). See, e.g., Clemens, J. C. et al. (2000) Proc. Natl. Sci. USA 97, 6499-6503; Billy, E. et al. (2001) Proc. Natl. Sci. USA 98, 14428-14433; Elbashir et al. (2001) Nature. 411(6836):494-8; Yang, D. et al. (2002) Proc. Natl. Acad. Sci. USA 99, 9942-9947, US 2003-0166282, 2003-0143204, 2004-0038278, and 2003-0224432.

Anti-sense agents can include, for example, from about 8 to about 80 nucleobases (i.e. from about 8 to about 80 nucleotides), e.g., about 8 to about 50 nucleobases, or about 12 to about 30 nucleobases. Anti-sense compounds include ribozymes, external guide sequence (EGS) oligonucleotides (oligozymes), and other short catalytic RNAs or catalytic oligonucleotides which hybridize to the target nucleic acid and modulate its expression. Anti-sense compounds can include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, e.g., under physiological conditions in the case of in vivo assays or therapeutic treatment or, in the case of in vitro assays, under conditions in which the assays are conducted.

Hybridization of antisense oligonucleotides with mRNA can interferes with one or more of the normal functions of mRNA. The functions of mRNA to be interfered with include all vital functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

Exemplary antisense compounds include DNA or RNA sequences that specifically hybridize to the target nucleic acid. The complementary region can extend for between about 8 to about 80 nucleobases. The compounds can include one or more modified nucleobases. Modified nucleobases may include, e.g., 5-substituted pyrimidines such as 5-iodouracil, 5-iodocytosine, and C5-propynyl pyrimidines such as C5-propynylcytosine and C5-propynyluracil. Other suitable modified nucleobases include N⁴—(C₁-C₁₂)alkylaminocytosines and N⁴,N⁴—(C₁-C₁₂)dialkylaminocytosines. Modified nucleobases may also include 7-substituted-8-aza-7-deazapurines and 7-substituted-7-deazapurines such as, for example, 7-iodo-7-deazapurines, 7-cyano-7-deazapurines, 7-aminocarbonyl-7-deazapurines. Examples of these include 6-amino-7-iodo-7-deazapurines, 6-amino-7-cyano-7-deazapurines, 6-amino-7-aminocarbonyl-7-deazapurines, 2-amino-6-hydroxy-7-iodo-7-deazapurines, 2-amino-6-hydroxy-7-cyano-7-deazapurines, and 2-amino-6-hydroxy-7-aminocarbonyl-7-deazapurines. Furthermore, N⁶—(C₁-C₁₂)alkylaminopurines and N⁶,N⁶—(C₁-C₁₂)dialkylaminopurines, including N⁶ methylaminoadenine and N⁶,N⁶-dimethylaminoadenine, are also suitable modified nucleobases. Similarly, other 6-substituted purines including, for example, 6-thioguanine may constitute appropriate modified nucleobases. Other suitable nucleobases include 2-thiouracil, 8-bromoadenine, 8-bromoguanine, 2-fluoroadenine, and 2-fluoroguanine. Derivatives of any of the aforementioned modified nucleobases are also appropriate. Substituents of any of the preceding compounds may include C₁-C₃₀ alkyl, C₂-C₃₀ alkenyl, C₂-C₃₀ alkynyl, aryl, aralkyl, heteroaryl, halo, amino, amido, nitro, thio, sulfonyl, carboxyl, alkoxy, alkylcarbonyl, alkoxycarbonyl, and the like.

Descriptions of other types of nucleic acid agents are also available. See, e.g., U.S. Pat. No. 4,987,071; U.S. Pat. No. 5,116,742; U.S. Pat. No. 5,093,246; Woolf et al. (1992) Proc Natl Acad Sci USA; Antisense RNA and DNA, D. A. Melton, Ed., Cold Spring Harbor Laboratory, Cold Spring Harbor, N.Y. (1988); 89:7305-9; Haselhoff and Gerlach (1988) Nature 334:585-59; Helene, C. (1991) Anticancer Drug Des. 6:569-84; Helene (1992) Ann. N.Y. Acad. Sci. 660:27-36; and Maher, L. J. (1992) Bioassays 14:807-15.

### Artificial Transcription Factors

Artificial transcription factors can also be used to regulate genes whose expression is altered in scleroderma, e.g., to increase the expression of a gene listed in Columns 2, 4, and 6 of Table 1 or to decrease expression of a gene list in Table 2. Artificial transcription factors can also be used to regulate genes that encode components of the Wnt pathway (e.g., to increase expression of negatively acting components (e.g., an sFRP, WIF, or Cerberus) or to decrease expression of positively acting components (e.g., a Wnt or Wnt receptor)). The protein can be designed or selected from a library. For example, the protein can be prepared by selection in vitro (e.g., using phage display, U.S. Pat. No. 6,534,261) or in vivo, or by design based on a recognition code (see, e.g., WO 00/42219 and U.S. Pat. No. 6,511,808). See, e.g., Rebar et al. (1996) Methods Enzymol 267:129; Greisman and Pabo (1997) Science 275:657; Isalan et al. (2001) Nat. Biotechnol 19-656; and Wu et al. (1995) Proc. Nat. Acad. Sci. USA 92:344 for, among other things, methods for creating libraries of varied zinc finger domains.

Optionally, the zinc finger protein can be fused to a transcriptional regulatory domain, e.g., an activation domain to activate transcription or a repression domain to repress transcription. The zinc finger protein can itself be encoded by a heterologous nucleic acid that is delivered to a cell or the protein itself can be delivered to a cell (see, e.g., U.S. Pat. No. 6,534,261. The heterologous nucleic acid that includes a sequence encoding the zinc finger protein can be operably linked to an inducible promoter, e.g., to enable fine control of the level of the zinc finger protein in the cell. Artificial zinc finger proteins can be administered directly, e.g., using a protein transduction domain, or by delivering a nucleic acid encoding the artificial zinc finger protein, e.g., using a gene therapy vector.

### Recombinant Protein Production

The nucleic acids encoding proteins that function as agents for the methods described herein may be operably linked to an expression control sequence in a vector in order to produce the protein recombinantly. General methods of expressing recombinant proteins are exemplified in Kaufman, Methods in Enzymology 185, 537-566 (1990), Sambrook & Russell, Molecular Cloning: A Laboratory Manual, 3^{rd} Edition, Cold Spring Harbor Laboratory, N.Y. (2001) and Ausubel et al., Current Protocols in Molecular Biology (Greene Publishing Associates and Wiley Interscience, N.Y. (1989). Examples of regulatory sequences that can be used to direct gene expression are described, for example, in Goeddel; Gene Expression Technology: Methods in Enzymology 185, Academic Press, San Diego, Calif. (1990).

Cells can be, for example prokaryotic (e.g., E. coli), yeast, plant, or mammalian. Exemplary mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK, Rat2, BaF3, 32D, FDCP-1, PC12, M1x or C2C12 cells. Transgenic animals (particularly mammals) can also be used to produce the recombinant protein (e.g., in milk).

### Treatments and Pharmaceutical Compositions

A therapeutic agent described herein can be provided as a pharmaceutical composition. Exemplary therapeutic agents include an agent that inhibits Wnt signaling an agent that inhibits IGF activity, an agent that decreases activity or expression a gene product listed in Columns 2, 4, and 6 of Table 1, or an agent that increases activity or expression of a gene product listed in Table 2.

The pharmaceutical composition may include a therapeutically effective amount of an agent described herein. A therapeutically effective amount is an amount effective, at dosages and for periods of time necessary, to achieve the desired therapeutic result or to prevent or delay onset of a disorder. A therapeutically effective amount of the composition may vary according to factors such as the disease state, age, sex, and weight of the individual, and the ability of the compound to elicit a desired response in the individual. A therapeutically effective amount is also one in which any toxic or detrimental effects of the composition is outweighed by the therapeutically beneficial effects. A therapeutically effective amount preferably modulates a measurable parameter, e.g., an indicia of scleroderma, e.g., to a statistically significant degree. The ability of a compound to inhibit a measurable parameter can be evaluated in an animal model system predictive of efficacy in a human disorder, using in vitro assays, e.g., an assay described herein, or using appropriate human trials. A variety of animal models of scleroderma can be used. Examples are described in Clark (2005) Curr Rheumatol Rep. 7(2): 150-5.

Particular effects mediated by an agent may show a difference that is statistically significant (e.g., P value<0.05 or 0.02). Statistical significance can be determined by any art known method. Exemplary statistical tests include: the Students T-test, Mann Whitney U non-parametric test, and Wilcoxon non-parametric statistical test. Some statistically significant relationships have a P value of less than 0.05 or 0.02. An increase or decrease can cause a qualitative or quantitative difference relative to a reference state, e.g., a statistically significant difference (e.g., P value<0.05 or 0.02).

Dosage regimens can be adjusted to provide the optimum desired response (e.g., a therapeutic response). For example, a single bolus may be administered, several divided doses may be administered over time or the dose may be proportionally reduced or increased as indicated by the exigencies of the therapeutic situation. It is possible to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

An exemplary, non-limiting range for a therapeutically effective amount of an agent described herein is 0.1-20 mg/kg, more preferably 1-10 mg/kg. Dosage values may vary with the type and severity of the condition to be alleviated. For any individual subject, specific dosage regimens can be adjusted over time according to the individual need and the professional judgment of the person administering or supervising the administration of the compositions. Accordingly, the dosage ranges set forth herein are only exemplary.

Subjects who can be treated include human and non-human animals, e.g., non-mammals (such as chickens, amphibians, reptiles) and mammals, such as non-human primates, mice, sheep, dogs, cows, pigs, etc.

An agent described herein may be used as a pharmaceutical composition when combined with a pharmaceutically acceptable carrier. Such a composition may contain, in addition to the agent and carrier, various diluents, fillers, salts, buffers, stabilizers, solubilizers, and other materials. Pharmaceutically acceptable carriers are non-toxic materials that does not interfere with the effectiveness of the biological activity of the active ingredient(s). The characteristics of the carrier typically depend on the route of administration.

In practicing the method of treatment or use, a therapeutically effective amount of an agent is administered to a subject, e.g., mammal (e.g., a human). The agent may be administered either alone or in combination with other therapies such as other treatments for fibrotic disorders, e.g., scleroderma. When co-administered with one or more agents, the agent may be administered either simultaneously with the second agent, or sequentially. If administered sequentially, the attending physician can decide on the appropriate sequence of administering the agent described herein with other agents.

Administration of an agent described herein can be carried out in a variety of ways, including, for example, oral ingestion, inhalation, or cutaneous, subcutaneous, or intravenous injection or administration. Topic administration can include direct application to a lesion, e.g., to sclerotic skin.

For oral administration, the agent can be in the form of a tablet, capsule, powder, solution or elixir. When administered in tablet form, the pharmaceutical composition may additionally contain a solid carrier such as a gelatin or an adjuvant. The tablet, capsule, and powder contain from about 5 to 95% of the agent or from about 25 to 90% of the agent. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of the agent, e.g., from about 1 to 50% or the agent.

To administer an agent, e.g., by intravenous, cutaneous or subcutaneous injection, the agent can be in the form of a pyrogen-free, parenterally acceptable aqueous solution. The preparation of such parenterally acceptable protein solutions, having due regard to pH, isotonicity, stability, and the like, is within the skill in the art. An exemplary pharmaceutical composition for intravenous, cutaneous, or subcutaneous injection can contain, in addition to the agent an isotonic vehicle such as sodium chloride injection, Ringer's injection, dextrose injection, dextrose and sodium chloride injection, lactated Ringer's injection, or other vehicle. The pharmaceutical composition may also contain stabilizers, preservatives, buffers, antioxidants, or other additive.

The amount of an agent to be delivered can depend upon the nature and severity of the condition being treated, and on the nature of prior treatments that the patient has undergone. The attending physician can decide the amount of agonist with which to treat each individual patient. Initially, for example, the attending physician can administer low doses of the agent and observe the patient's response. Larger doses of the agent may be administered until the optimal therapeutic effect is obtained for the patient, and at that point the dosage is not generally increased further, or by monitoring one or more symptoms.

In the case of an agent that is an immunoglobulin (e.g., a full length antibody), an exemplary pharmaceutical compositions may contain about 0.1 μg to about 10 mg of the immunoglobulin agent per kg body weight. For example, useful dosages can include between about 10 μg−1 mg, 0.1-5 mg, and 3-50 mg of the agent per kg body weight.

The duration of therapy using the pharmaceutical composition can vary, depending on the severity of the disease being treated and the condition and potential idiosyncratic response of each individual patient. The duration of each application of the agent can be, e.g., in the range of 12 to 24 hours of continuous intravenous administration. The attending physician can decide on the appropriate duration of intravenous therapy using a pharmaceutical composition described herein.

With respect to agents that are proteins or nucleic acids, the disease or disorder can also be treated or prevented by administration or use of polynucleotides encoding such proteins (such as, for example, in gene therapies or vectors suitable for introduction of DNA). The polynucleotides that encode an agent or that provide a nucleic acid agent activity can be inserted into vectors and used as gene therapy vectors. Gene therapy vectors can be delivered to a subject by, for example, intravenous injection, local administration (see U.S. Pat. No. 5,328,470), injection (e.g., US 2004-0030250 or 2003-0212022) or stereotactic injection (e.g., Chen et al. Proc. Natl. Acad. Sci. USA 91:3054-3057, 1994). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

### Diagnostics

Information about the expression of one or more genes described herein (e.g., one or more genes listed in Table 1 or 4) can be used to evaluate a subject or a culture. The subject can be evaluated, e.g., to determine risk for scleroderma or other fibrotic disorder, e.g., to predict whether the subject is likely to get the disorder prior to its onset, or to determine whether the subject has the disorder. The subject can be an adult, child, fetus, or gamete. The evaluation can be made by comparing a value indicative of expression (e.g., qualitative or quantitative values) in the subject to a reference, e.g., a reference, e.g., a reference obtained from a control (e.g., a healthy subject) or a reference that is a statistical representation of a cohort (e.g., cohort of diseased or healthy subjects).

The subject can be evaluated prior to, during, or after a treatment, e.g., to determine efficacy of the treatment. Information from the evaluation can be used to modify the treatment, e.g., to increase or decrease the dose of an agent on subsequent administrations. Values indicative of expression (e.g., qualitative or quantitative values) can be compared to a reference, e.g., a reference for the subject obtained prior to an initial treatment, a reference for the subject obtained prior to disease onset, or other reference, e.g., reference obtained from a control (e.g., a healthy subject) or a statistical representation of a cohort (e.g., cohort of diseased or healthy subjects).

The evaluation can include evaluate a single gene, e.g., a gene encoding WIF1, IGFBP, or collagen, e.g., collagen XI. The evaluation can also include evaluating expression of multiple genes, e.g., a plurality of genes described herein. Information about the expression of multiple genes can be used to provide a gene expression profile.

An exemplary scheme for producing and evaluating profiles is as follows. Nucleic acid is prepared from a sample, e.g., a sample of interest and hybridized to an array, e.g., with multiple addresses. Hybridization of the nucleic acid to the array is detected. The extent of hybridization at an address is represented by a numerical value and stored, e.g., in a vector, a one-dimensional matrix, or one-dimensional array. The vector x {xₐ, x_{b} . . . } has a value for each address of the array. For example, a numerical value for the extent of hybridization at a first address is stored in the variable xₐ. The numerical value can be adjusted, e.g., for local background levels, sample amount, and other variations. Nucleic acid is also prepared from a reference sample and hybridized to an array (e.g., the same or a different array), e.g., with multiple addresses. The vector y is construct identically to vector x. The sample expression profile and the reference profile can be compared, e.g., using a mathematical equation that is a function of the two vectors. The comparison can be evaluated as a scalar value, e.g., a score representing similarity of the two profiles. Either or both vectors can be transformed by a matrix in order to add weighting values to different nucleic acids detected by the array.

The expression data can be stored in a database, e.g., a relational database such as a SQL database (e.g., Oracle or Sybase database environments). The database can have multiple tables. For example, raw expression data can be stored in one table, wherein each column corresponds to a nucleic acid being assayed, e.g., an address or an array, and each row corresponds to a sample. A separate table can store identifiers and sample information, e.g., the batch number of the array used, date, and other quality control information.

Nucleic acids that are similarly regulated can be identified by clustering expression data to identify coregulated nucleic acids. Nucleic acids can be clustered using hierarchical clustering (see, e.g., Sokal and Michener (1958) Univ. Kans. Sci. Bull. 38: 1409), Bayesian clustering, k-means clustering, and self-organizing maps (see, Tamayo et al. (1999) Proc. Natl. Acad. Sci. USA 96: 2907).

Expression profiles obtained from nucleic acid expression analysis on an array can be used to compare samples and/or cells in a variety of states as described in Golub et al. ((1999) Science 286: 531). For example, multiple expression profiles from different conditions and including replicates or like samples from similar conditions are compared to identify nucleic acids whose expression level is predictive of the sample and/or condition. Each candidate nucleic acid can be given a weighted “voting” factor dependent on the degree of correlation of the nucleic acid's expression and the sample identity. A correlation can be measured using a Euclidean distance or a correlation coefficient, e.g., the Pearson correlation coefficient.

The similarity of a sample expression profile to a predictor expression profile (e.g., a reference expression profile that has associated weighting factors for each nucleic acid) can then be determined, e.g., by comparing the log of the expression level of the sample to the log of the predictor or reference expression value and adjusting the comparison by the weighting factor for all nucleic acids of predictive value in the profile.

As described above, information about gene expression can include information obtained by evaluating mRNA levels or by evaluating protein levels.

### Fibrotic Disorders

In addition to scleroderma, the methods described herein (particularly the therapeutic methods) may also be generally applicable to other fibrotic disorders. Fibrotic disorders include fibrosis of an internal organ, a dermal fibrosing disorder, and fibrotic conditions of the eye.

Fibrosis of internal organs (e.g., liver, lung, kidney, heart blood vessels, gastrointestinal tract), occurs in disorders such as pulmonary fibrosis, myelofibrosis, liver cirrhosis, mesangial proliferative glomerulonephritis, crescentic glomerulonephritis, diabetic nephropathy, renal interstitial fibrosis, renal fibrosis in patients receiving cyclosporin, and HIV associated nephropathy. Dermal fibrosing disorders include, e.g., scleroderma, morphea, keloids, hypertrophic scars, familial cutaneous collagenoma, and connective tissue nevi of the collagen type. Fibrotic conditions of the eye include conditions such as diabetic retinopathy, post-surgical scarring (for example, after glaucoma filtering surgery and after cross-eye surgery), and proliferative vitreoretinopathy.

Additional fibrotic conditions include: rheumatoid arthritis, diseases associated with prolonged joint pain and deteriorated joints; progressive systemic sclerosis, polymyositis, dermatomyositis, eosinophilic fascitis, morphea, Raynaud's syndrome, and nasal polyposis.

### Example

The following exemplary methods were used to identify changes in gene expression associated with scleroderma.

### Patients and Controls

Patients who (i) fulfilled the preliminary classification criteria for scleroderma of the American Rheumatism Association (ARA now the American College of Rheumatology) and (ii) had diffuse cutaneous disease according to the classification of LeRoy et al (J. Rheumatol. 1988 February; 15(2):202-5) were selected. Consecutive outpatients with early (less than 3 years from the onset of the first non-Raynaud's phenomenon scleroderma disease manifestation) and diffuse disease were chosen in an attempt to diminish heterogeneity of disease expression. To be eligible for the study, patients could not have been on doses of prednisone greater or equal to 20 mg in the 4 weeks prior to the biopsy, nor on other immunosuppressive treatment within this time period.

Healthy controls were chosen on the basis of lack of Raynaud's phenomenon by history, lack of a diagnosis of a systemic autoimmune disease, and willingness to participate. Control subjects who did not use glucocorticoids or immunosuppressive agents were selected. Characteristics of the subjects are summarized in Table 3, below.

TABLE 3
Characteristics Of The Study Population That
Was Used For Gene Expression Analysis

Total

| | Patient | Control |
|---|---|---|
| Female:Male | 7:2 | 5:4 |
| Mean Age | 45.7 ± 8.5 | 46.9 ± 8.8 |
| Caucasian:African American | 7:2 | 6:3 |
| Diffuse:Limited | 8:1 | NA |

Two 3-mm punch biopsies were taken from the same distal forearm (involved skin in the case of the scleroderma patients) of each subject in a side-by-side fashion. One biopsy specimen was immediately placed in RNALater® (Ambion) and stored at 4° C. prior to RNA preparation. RNA was prepared within 15 days of tissue harvest. The other biopsy piece was placed in culture.

### Fibroblast Culture Conditions

Biopsy tissue was rinsed several times with antibiotic-antimycotic solution (Life Technologies Cat. No. 15240-062). Tissue was then placed in 1 ml of collagenase solution and incubated for 24 hours at 37° C. Collagenase solution contained 0.25% collagenase type I (Sigma) and 0.05% DNaseI (Sigma) in Dulbecco's modified Eagle's medium (DMEM) with 20% fetal bovine serum (FBS) (HyClone, Logan, Utah). The entire 1 ml was mixed together with 5 ml of media (DMEM+20% FCS), then plated into 25 cm2 flask, and left undisturbed for 48 hours at 37° C. in a 5% CO₂ atmosphere. The resulting confluent culture was then designated passage-1 or P1. Cells were then split 1:4 to generate 4×25 cm2 flasks (P2). Two flasks at passage 4 were trypsinized, the trypsin was neutralized using soybean trypsin inhibitor, and cell pellets were washed twice with PBS prior to addition of RNAlater® (Ambion). Cell pellets were shipped frozen on dry ice by overnight delivery and stored frozen at −80° C. prior to RNA preparation. A 3 mm biopsy stored in RNAlater® according to manufacturers instructions gave ample material for a labeling and hybridization reaction without amplification. Twenty-eight biopsies were processed. Most biopsies gave yields in excess of 2 μg RNA and were hybridized.

### Total RNA Purification

Skin biopsies were removed from RNAlater® solution (Ambion, Austin, Tex.), placed in a weighing dish containing 1 ml of TRIzol® reagent (Invitrogen, Carlsbad, Calif.), minced using a razor blade, and poured into a 2 ml tube. RNAlater® was removed from fibroblast pellets, which were then resuspended in 1 ml of TRIzol® reagent. Fibroblasts and biopsies were homogenized using a PowerGen™ 125 (Fisher Scientific, Hampton, N.H.) for 2 to 5 minutes at top speed. Total RNA was extracted from TRIzol® according to manufacturer's protocol. Biopsy extraction included a centrifugation step to treat samples with high content of fat and extracellular materials. Total RNA was resuspended in 100 μL of water and further purified using an RNEASY MINI™ column (Qiagen, Valencia, Calif.) according to manufacturer's protocol.

### Probe Labeling, Hybridization and Scanning

Sample labeling, hybridization, and staining were carried out according to the Eukaryotic Target Preparation protocol in the Affymetrix Technical Manual (701021 rev. 4) for GENECHIP® Expression Analysis (Affymetrix, Santa Clara, Calif.). In summary, 1 to 5 μg of purified total RNA was used in a 20 μL first strand reaction with 200 U SuperScript II (Invitrogen) and 0.5 μg (dT)-T7 primer in 1× first strand buffer (Invitrogen) with a 42° C. incubation for 1 hour. Second strand synthesis was carried out by the addition of 40 U E. coli DNA Polymerase, 2 U E. coli RNase H, 10 U E. coli DNA Ligase in 1× second strand buffer (Invitrogen) followed by incubation at 16° C. for 2 hrs.

The second strand synthesis reaction was purified using the GENECHIP® Sample Cleanup Module according to the manufacturer's protocol (Affymetrix). Purified cDNA was amplified and biotinylated using BIOARRAY HIGHYIELD™ RNA Transcript Labeling Kit (Enzo Life Sciences, Farmingdale, N.Y.) according to manufacturer's protocol. 15 μg of labeled cRNA was fragmented and resuspended in 300 μL 1× hybridization buffer containing 100 mM MES, 1 M [Na+], 20 mM EDTA, 0.01% TWEEN® 20, 0.5 mg/mL acetylated BSA, 0.1 mg/mL herring sperm DNA, control oligo B2, and eukaryotic control transcripts. The labeled material was applied to a Human Genome 133A GENECHIP® (Affymetrix). Hybridized arrays were washed and stained on a GENECHIP® Fluidics Station 450 and visualized using a GENECHIP® Scanner 3000.

### Quantitative RTPCR

First-Strand cDNA was synthesized from RNA using SUPERSCRIPT III PLATINUM™ Two-Step qRT-PCR Kit (Invitrogen). For each reaction 1 uL of RNA containing 100 ng-lug was used. RT-PCR reactions were set up in Optical 96-Well Reaction Plates (Applied Biosystems) using TAQMAN™ Gene Expression Assays Protocol for 50 μL Reactions. Concentration of cDNA was determined by spectroscopy using a BIOPHOTOMETER™ (Eppendorf). For all reactions 40 ng of cDNA was used. RT-PCR thermal profile: 3:00 @ 95°; then 55 cycles of 0:15 @ 95°, 1:00 @ 56°. RT-PCR data was collected using an Mx300P™ PCR machine (Stratagene) and analyzed using Mx3000P™ software (Stratagene).

### Array Analysis

Array data in the form of CEL files were imported into BRB array tools. Biopsy data and fibroblast data were imported and normalized independently using RMA algorithm. Datasets normalized using the MAS5 algorithm implemented in R were used when comparisons between fibroblasts and biopsies were necessary. Class comparisons and class predictions were carried out using the BRB software package (available from Dr. Richard Simon and Amy Peng Lam, National Cancer Institute, Bethesda Md.).

### Immunohistochemistry

Specimens were analyzed from individuals with similar age and history as those used for the gene array experiments. Immunohistochemistry was performed according to manufacturer's instructions using diaminobenzidine (DAB) as chromogen.

Comparison of Normal and SSC biopsies

Unsupervised (agglomerative) clustering of all the samples demonstrated that the scleroderma phenotype was the dominant influence on expression profile, and was not confounded by patient sex, age, race, or origin of the biopsy. Class comparisons on all 22000 qualifiers between normal and scleroderma biopsies showed 1839 qualifiers distinguishing normal skin from scleroderma at a p value≦0.01, n=17 (unpaired T-tests, with random variance model and a false discovery rate<0.1), of which the 50 most significant by p value are shown below: WIF1, CTGF, NNMT, PRSS23, LBH, SFRP4, CHN1, LUM, SERPINE2, D2S448, THBS1, ABHD6, LOXL1, RAB31, IGF1, COMP, RAB31, IFITM2, FKBP11, ASPN, IGFBP6, IGFBP3, THY1, CDH11, THY1, OAS1, GARP, SERPINE1, NOX4, HCA112, ADCY2, RELB, SGCG, GALNT10, TNFRSF6B, COL6A3, FN1, TP5313, COL4A2, ADAM12, CLDN8, CAPN3, IGFBP3, and TNFSF4.

### Comparison of Normal and SSc Fibroblasts and Relationship to the Biopsies

Class comparisons between normal and scleroderma fibroblasts showed 223 qualifiers distinguishing normal from scleroderma at p<0.01. Of these, 105 were up-regulated in scleroderma, and 118 down-regulated. The intersection between qualifiers dysregulated in biopsies and those in fibroblasts (26, of which 9 are discordant, 17 are concordant) was far greater than that which would be seen by chance (p<0.02, Fisher's Exact test of observing an intersection of that many or more by random sampling of gene lists from a pool of 22000 qualifiers.), and included a large proportion of extracellular matrix genes, including collagen VIII, fibulin I, fibrillin 2, and decorin. Interestingly, a subset of these showed inverse regulation in the context of the biopsy and the fibroblasts, notably ephrin B2.

The extent of change in gene expression levels is complicated by the fact that each biopsy contains maintains multiple cell types. A particular change in gene expression may occur in only one of the many cell types in the biopsy. As an approximation, we considered genes that were up-regulated at least five fold in cultured fibroblasts as likely to be of fibroblast origin in the biopsy, and, conversely, genes that were down-regulated at least five-fold in fibroblasts relative to the biopsy were unlikely to be of fibroblast origin in the biopsy. Genes between these two extremes of differential expression were considered indeterminate in source.

In order that genes influenced by the disease process would not be averaged out, class comparisons of scleroderma biopsies to scleroderma fibroblasts were made independently of control biopsies versus control fibroblasts, and a union made of the five-fold downregulated (or five-fold upregulated) qualifiers. These lists were intersected with the class comparison of scleroderma biopsies versus control biopsies. Thus, for example, qualifiers q dysregulated in the fibroblast component were found such that (((q_{fibrossc}>5×q_{biopsyssc}, p<0.01) OR (q_{fibron1}>5×q_{biopsy}, p<0.01)) AND (q_{biopsyn1}< >q_{biopsyssc}, p<0.01)). The top 25 by p value of “probable fibroblast” and “probable non-fibroblast” genes are shown below.

Nonfibroblast: WIF1, SFRP4, ABHD6, IGF1, COMP, OAS1, ADCY2, CLDN8, CAPN3, C1QR1, PECAM1, IGF1, IFI27, ACADL, ZNF204, C1QB, CD14, RALGPS1, CCL19, A2M, IL1F7, GPM6A, ALDH5A1, C4A, CD209, and ERBB3.

Fibroblast: CTGF, NNMT, PRSS23, CHN1, SERPINE2, D2S448, THBS1, LOXL1, IGFBP3, THY1, CDH11, THY1, GARP, SERPINE1, NOX4, ADAM12, IGFBP3, D2S448, CDH11, SERPINE1, DAB2, TIMP1, ANXA6, DAB2, ADAM12, and FN1.

We created an exemplary list of genes that are useful as an expression profile for distinguishing normal from scleroderma fibroblasts and for distinguishing scleroderma biopsies from controls. Biopsies, unlike cultured fibroblasts, may reflect actual expression of cells in a subject as the evaluated cells in a biopsies are not given the opportunity for cellular adjustment that may result during culturing. We performed a class comparison between scleroderma fibroblasts and normal fibroblasts at p<0.01. The 223 member qualifier list that resulted from this comparison was used to generate a classifier for the biopsies. Leave-one-out cross validation analysis selected a subset of 26 genes, which were successfully distinguished scleroderma from normal biopsies according to multiple models. Fifteen genes could be matched to qualifiers in the HU95A chip. Examples of these genes are: ADSL, C1QA, MX2, COL8A1, ICAM1, C7orf19, OAS1, HS3ST3A1, IFI16, ANGPT2, DAP, NINJ2, SLC16A3, TNIP2, SDC3, FBN2, FZD2, LOC51334, MTCP1, PAQR6, DCN, ABCA6, LOC114977, PLP1, EFNB2, and FBLN1.

Changes in the population or activity of immune cells in biopsies can be detected using T and B cell markers. Examples of T cell markers include CD3, CD4, and CD28, the monocyte markers CD14, CD163, and CD11b, the antigen presenting cell co-stimulatory protein B7-2 (CD86). Examples of B cell markers include CD83, CD84, and Ig kappa. CD3 (T cell) counts were not obviously different on immunohistochemistry between control and scleroderma samples, and CD20 (B cell) stains showed very few cells on all specimens. This suggests that even very small numbers of lymphocytes can be detected by gene expression profiling. The gene expression analysis detected an immune cell signature from B cells, T cells and macrophages, consistent with the abundant auto-antibodies characteristic of scleroderma.

We observed that the expression of chondrogenesis associated collagens, such as collagen XI and collagen X was increased, as was that of the chondrogenesis associated protein (COMP), the nonfibrillar collagen IV, the network forming collagens (collagens X and VIII), the endothelial basement membrane collagen XV, and BMP1 (a collagen and biglycan processing enzyme). Significantly, the expression of collagen XI is increased even more than that of other collagens: ˜5-fold, relative to ˜2-fold for most other collagens. Collagen V forms fibrils with collagen I to control fibril diameter and collagen XI forms fibrils with collagen II in an analogous fashion. Collagen XI is found in association with collagen I in fibrocartilage of the intervertebral disc of normal individuals, as well as in the embryonic tendon. Thus, the fibrotic response resembles the specific developmental program for fibrocartilage. Interestingly, in a study of lung fibroblast responses to TGFβ, collagen IV was the only collagen significantly dysregulated.

The small leucine rich family of proteins (SLRPs) also show some characteristic alterations in scleroderma (Table 4). Decorin is down regulated in scleroderma, and may have TGFβ antagonistic properties of its own. On the other hand, biglycan, versican and lumican are up regulated, as is asporin, a homolog of decorin. The potential regulatory roles of the SLRPs in matrix assembly are beginning to be dissected. While all of them are associated with collagen fibrils, each may be synthesized by different subsets of cell types. All of these proteins have generally inhibitory effects on collagen fibril diameter and on fibroblast proliferation.

TABLE 4

| Upregulated in Ssc | Downregulated in SSc | Not regulated |
|---|---|---|
| Collagens, particularly 11, 10, | Decorin | Fibromodulin |
| 5, and 4 | | |
| Thrombospondin | Tenascin X | |
| Tenascin C | Laminin | |
| Lumican | Fibulin | |
| Fibronectin | | |
| Biglycan | | |
| Versican | | |
| Aggrecan | | |
| Fibrillin 2 | | |

The TGFβ pathway is activated in cultured scleroderma fibroblasts. The expression of many gene expression targets of TGFβ was increased in scleroderma biopsies. Many of these targets are no longer differentially expressed in explanted fibroblasts, notably the bulk of the collagens, which suggests that the profibrotic drivers are from cell types which do not persist in fibroblast culture. Transcripts for TGFβ itself were not increased in scleroderma biopsies, suggesting that increased TGFβ signal is due to increased activation of latent TGFβ protein.

Thrombospondin I, an activator of latent TGFβ, is significantly upregulated in the scleroderma biopsies. Studies have suggested that the subset of pulmonary fibroblasts which are Thy-1 positive are in fact TGFβ insensitive due to failure of TGFβ activation. Expression of Thy-1 was increased in scleroderma skin along with thrombospondin I, suggesting that this relationship does not hold in dermal fibrosis.

Changes seen in the Wnt pathway encompass many gene products more likely to be related to cell types other than fibroblasts, including decreased expression of WIF1, frizzled related protein, and frizzled homolog 7, as well as increased expression of secreted frizzled-related protein 4. These changes are consistent with a general increase in Wnt signalling. Interestingly, a Wnt regulatory system has been shown to be active in mesenchymal stem cells in culture. There was a decrease in Dkk and a reciprocal increase in Wnt5A associated with a deceleration in cell growth.

Changes in gene expression of members of the CCN family were also detected. The CCN family includes connective tissue growth factor (CTGF, CCN2), a putative downstream target of TGFβ and a profibrotic cytokine. CCN2 is up regulated in scleroderma skin and scleroderma fibroblasts (see also). Expression of Cyr61 (CCN1), which provides integrin dependent promigratory stimuli to fibroblasts and vascular smooth muscle cells is also increased in scleroderma, while expression of WISP2 (CCN5) is decreased. Loss of Cyr61 is associated with differentiation of mesenchymal stem cells into any daughter lineage. Thus its up regulation may reflect expansion of an uncommitted fibroblast phenotype. Reciprocal regulation of WISP2 and CTGF has been noted in the fibroblast response to TGFβ.

With the observations above, profiling data from both biopsies and fibroblasts enabled us to make some informed guesses about the tissue type contribution of different genes in scleroderma. For example, increased expression of monocyte/macrophage genes CD14, TLR 1 and 2, integrins β2, αX, and αM appear to be associated with increased expression of IL6, IL16, and CXCL3, all of which may be synthesized by fibroblasts. Similarly, a decrease in expression of IGFBP 5 and 6 and WISP2, likely from a nonfibroblast source, and a reciprocal increase in expression of IGFBP 3 and 4, both presumably in fibroblasts, as well as CTGF, PAI-1, and C1q, the latter from a non-fibroblast source were observed. Accordingly, in scleroderma lesions, there may be a loss of inhibitory signals from a non-fibroblast cells and concomitant increase in profibrotic behavior by fibroblasts. It is possible that the downregulation of some epithelial-derived genes is due to loss of epithelial adnexae in sclerodermatous skin. However, while there was a two fold decrease in expression of keratin 15, associated with early hair follicle “bulge” cells, no other markers of follicles, such as keratin 19 and the hair keratins, were significantly or consistently altered in the scleroderma biopsies.

The comparison of changes to fibroblast cultures in scleroderma to changes in primary biopsies in scleroderma enabled dissecting fibroblast driven from non-fibroblast driven behavior. While there is a robust set of genes whose expression distinguishes scleroderma from normal fibroblasts; there is a reduced number of genes whose expression distinguishes scleroderma biopsies from control at the same level of significance (223 vs. 1839 at p=0.01). For example, CTGF, well known as a fibrosis driver downstream of TGFβ, as well as Thy-1, are upregulated in scleroderma biopsies relative to normal controls, but in the cultured fibroblasts expression of these genes does not clearly correlate with disease. At the same time, matrix targets of CTGF and TGFβ, such as some of the collagens and fibronectin, are upregulated in the scleroderma fibroblasts. Thus, while the fibroblasts show alterations in gene expression that are a signature of the disease, they are likely to act at the end of a chain of events initiated by another cell type. Possible originators of this process may include the endothelial cell or its partner the pericyte, both targets of scleroderma mediated injury. In culture, however, disease-driving cells may be diluted out as fibroblasts proliferate in the culture system, leading to reversion of the fibroblast behavior.

A list of exemplary genes whose expression is significantly altered is provided below:

TABLE 1

| COLUMN 1 | COLUMN 2 | COLUMN 3 | COLUMN 4 | COLUMN 5 | COLUMN 6 |
|---|---|---|---|---|---|
| Upregulated in | Downregulated in | Upregulated in | Downregulated in | Upregulated in | Downregulated in |
| scleroderma | Scleroderma | scleroderma | Scleroderma | scleroderma | Scleroderma |
| 7h3 | ABCA12 | 7h3 | ABCA12 | 7h3 | ABHD5 |
| A2M | ABCA3 | A2M | ABCA3 | A2M | ABHD6 |
| ACP2 | ABCA5 | ACTG1 | ABCA5 | ACTG1 | ACAD8 |
| ACTB | ABCA6 | ADAM12 | ABCA6 | ADAMTS6 | ACADL |
| ACTG1 | ABCB1 /// | ADAM19 | ABCB1 /// | ADSL | ACSL1 |
| ADAM12 | ABCB4 | ADAMDEC1 | ABCB4 | AGTRL1 | ADCY2 |
| ADAM19 | ABHD5 | ADAMTS2 | ABHD5 | ALOX5 | ADPN |
| ADAMDEC1 | ABHD6 | ADAMTS6 | ABHD6 | AP2B1 | AGXT |
| ADAMTS2 | ABLIM1 | ADCYAP1 | ACAD8 | APOL2 | AKAP1 |
| ADAMTS6 | ACAD8 | ADORA3 | ACADL | APOL6 | AKR1C1 /// |
| ADCYAP1 | ACADL | ADRBK2 | ACADSB | ARHGAP8 | AKR1C2 |
| ADORA3 | ACADSB | ADSL | ACSL1 | ARHGDIB | AKR1C2 |
| ADRBK2 | ACSL1 | AGC1 | ACVR1B | ASPN | ALAD |
| ADSL | ACVR1B | AGTRL1 | ADCY2 | ASRGL1 | ALOXE3 |
| AEBP1 | ADCY2 | ALDH1B1 | ADD3 | ATP8B2 | ANKH |
| AGC1 | ADD1 | ALOX5 | ADPN | ATXN7L1 /// | ANKRA2 |
| AGTRL1 | ADD3 | ALOX5AP | AGTR1 | ATXN7L4 | ANKRD27 |
| AIF1 | ADPN | ANGPT2 | AGXT | B4GALT5 | ARHGAP19 |
| AKAP2 | AGTR1 | AOAH | AHNAK | BGN /// | ARHGEF10 |
| ALDH1B1 | AGXT | AP2B1 | AKAP1 | SDCCAG33 | ARL10C |
| ALOX5 | AHNAK | APC | AKR1C1 /// | BICC1 | ARMCX1 |
| ALOX5AP | AIM1 | APOBEC3B | AKR1C2 | BM039 | ATP5A1 |
| ANGPT2 | AKAP1 | APOL2 | AKR1C2 | C10orf3 | ATP5L |
| ANGPTL2 | AKR1C1 /// | APOL6 | ALAD | C11orf24 | ATP6V0A4 |
| ANXA5 | AKR1C2 | ARHGAP4 | ALDH2 | C13orf18 | ATPIF1 |
| ANXA6 | AKR1C2 | ARHGAP8 | ALDH3A2 | C16orf30 | BBS1 |
| AOAH | ALAD | ARHGDIB | ALDH5A1 | C19orf22 | BCAN |
| AP2B1 | ALDH2 | ARRB2 | ALOX12 | C1orf33 | BCL11B |
| APC | ALDH3A2 | ARSA | ALOXE3 | C1QA | BMP2 |
| APOBEC3B | ALDH5A1 | ARSB | AMT | C1QR1 | BRP44L |
| APOL2 | ALDH9A1 | ASPN | ANG | C20orf103 | C10orf57 |
| APOL6 | ALOX12 | ASRGL1 | ANK3 | C20orf39 | C14orf131 |
| ARF4 | ALOXE3 | ATP8B2 | ANKH | C3 | C14orf2 |
| ARHGAP4 | AMT | ATXN7L1 /// | ANKMY2 | C6orf97 | C14orf92 |
| ARHGAP8 | AMY2B | ATXN7L4 | ANKRA2 | C7orf10 | C18orf11 |
| ARHGDIB | ANG | B4GALT5 | ANKRD27 | CALD1 | C18orf9 |
| ARPC1B | ANK3 | BASP1 | ANKRD28 | CALM3 | C1orf35 |
| ARRB2 | ANKH | BCL3 | ANXA2 | CARHSP1 | C1QDC1 |
| ARSA | ANKMY2 | BGN /// | APP | CASP2 | C20orf111 |
| ARSB | ANKRA2 | SDCCAG33 | AQP9 | CD209 | C20orf38 |
| ASPN | ANKRD27 | BICC1 | AR | CD28 | C20orf42 |
| ASRGL1 | ANKRD28 | BICD1 | ARHGAP19 | CD4 | C9orf55 |
| ATP8B2 | ANXA2 | BM039 | ARHGEF10 | CD84 | CAB39L |
| ATXN7L1 /// | APP | BMP1 | ARHGEF12 | CD86 | CABC1 |
| ATXN7L4 | APPBP2 | BRCA2 | ARHGEF9 | CDC6 | CAT |
| B4GALT5 | AQP9 | C10orf3 | ARHI | CECR1 | CCNG2 |
| BASP1 | AR | C11orf24 | ARIH1 | CENTA2 | CD9 |
| BCL3 | ARHGAP19 | C13orf18 | ARL10C | CHD7 | CDC16 |
| BF | ARHGEF10 | C16orf30 | ARL4A | CINP | CDC5L |
| BGN | ARHGEF12 | C19orf10 | ARL6IP /// | CKAP1 | CDK5RAP1 |
| BGN /// | ARHGEF9 | C19orf22 | RPS15A | CKIP-1 | CELSR1 |
| SDCCAG33 | ARHI | C1orf33 | ARMCX1 | CLN2 | CGI-143 |
| BICC1 | ARIH1 | C1orf38 | ARMCX6 | CNN2 | CGI-41 |
| BICD1 | ARL10C | C1QA | ASCC3L1 | CNTNAP1 | CHCHD2 |
| BM039 | ARL4A | C1QR1 | ASGR1 | COL1A1 | CHN2 |
| BMP1 | ARL6IP /// | C20orf103 | ATP1B3 | COL5A1 | CLCA4 |
| BRCA2 | RPS15A | C20orf39 | ATP2B4 | COL5A2 | CLDN1 |
| BST2 | ARMCX1 | C3 | ATP5A1 | COL6A1 | COX15 |
| C10orf10 | ARMCX6 | C3AR1 | ATP5H | COL8A1 | COX7C |
| C10orf3 | ASCC3L1 | C4A | ATP5L | COL8A2 | CPE |
| C11orf24 | ASGR1 | C6orf32 | ATP6V0A4 | CORO1B | CRBN |
| C13orf18 | ATP1B3 | C6orf97 | ATP7A | COTL1 | CRYZL1 |
| C16orf30 | ATP2B4 | C7orf10 | ATPIF1 | CRN7 | CUL3 |
| C19orf10 | ATP5A1 | C7orf19 | ATRN | CSGlcA-T | CYP39A1 |
| C19orf22 | ATP5H | CALD1 | B3GNT6 | CTSB | CYP3A5 |
| C1orf29 | ATP5J | CALM3 | B4GALT3 | CXCL10 | CYP4F3 |
| C1orf33 | ATP5L | CAPNS1 | B4GALT4 | CYLD | DAF |
| C1orf38 | ATP5O | CARHSP1 | BAT8 | DACT1 | DCN |
| C1QA | ATP6V0A4 | CASP2 | BBS1 | DAPK3 | DDR1 |
| C1QB | ATP6V1H | CCNB2 | BCAN | DEPDC6 | DKFZp434C0328 |
| C1QR1 | ATP7A | CCND3 | BCAR3 | DKFZP434B044 | DKFZp761P211 |
| C1R | ATP9A | CD209 | BCKDHB | DKFZP434H132 | DLG3 |
| C1S | ATPIF1 | CD28 | BCL11B | DKFZP434P211 | DNCH2 |
| C2 | ATRN | CD37 | BCL2L2 | DKFZp762C186 | DOK4 |
| C20orf103 | AUH | CD4 | BDNF | DKFZp762E1312 | DSG2 |
| C20orf39 | B3GNT6 | CD83 | BEXL1 | DNM3 | DVL1 |
| C3 | B4GALT3 | CD84 | BLCAP | DPYSL2 | EBP |
| C3AR1 | B4GALT4 | CD86 | BLMH | DPYSL3 | ECHDC3 |
| C4A | BAT8 | CDC14B | BMP2 | EFEMP2 | EDD |
| C5orf13 | BBS1 | CDC6 | BRP44L | EFHD2 | EFHC2 |
| C6orf32 | BCAN | CDC7 | BTBD3 | ERP70 | EGF |
| C6orf97 | BCAR3 | CECR1 | C10orf57 | FAD104 | EIF2B1 |
| C7orf10 | BCKDHB | CENTA2 | C12orf22 | FAP | EIF2C1 |
| C7orf19 | BCL11B | CFLAR | C14orf1 | FARS1 | eIF3k |
| CALCRL | BCL2L2 | CH25H | C14orf124 | FCGR2A | EIF3S6IP |
| CALD1 | BDNF | CHC1 | C14orf131 | FGF13 | ELL3 |
| CALM3 | BEXL1 | CHD7 | C14orf132 | FKBP11 | ELOVL4 |
| CALU | BLCAP | CHN1 | C14orf2 | FLJ10292 | EMX2 |
| CAPNS1 | BLMH | CINP | C14orf92 | FLJ10385 | EPB41L4B |
| CARHSP1 | BMP2 | CKAP1 | C18orf11 | FLJ10647 | EPHX2 |
| CASP2 | BRP44L | CKIP-1 | C18orf9 | FLJ10815 | FAHD2A |
| CCL13 | BTBD3 | CLN2 | C1orf21 | FLJ11259 | FBXO42 |
| CCL18 | C10orf116 | CMKLR1 | C1orf35 | FLJ12438 | FBXO9 |
| CCL19 | C10orf57 | CNN2 | C1QDC1 | FLJ12439 | FGFR1 |
| CCL2 | C12orf22 | CNTNAP1 | C20orf111 | FLJ13725 | FH |
| CCL8 | C14orf1 | COL10A1 | C20orf38 | FLJ22175 | FHL1 |
| CCNB2 | C14orf124 | COL1A1 | C20orf42 | FLJ23221 | FLJ10415 |
| CCND3 | C14orf131 | COL4A4 | C21orf107 | FLJ23556 | FLJ10521 |
| CD14 | C14orf132 | COL5A1 | C6orf130 | FLJ30656 | FLJ10769 |
| CD163 | C14orf2 | COL5A2 | C9orf55 | FLNA | FLJ10847 |
| CD209 | C14orf92 | COL6A1 | CA11 | FLRT2 | FLJ10948 |
| CD28 | C18orf11 | COL8A1 | CAB39L | FMO3 | FLJ10986 |
| CD37 | C18orf9 | COL8A2 | CABC1 | FOLH1 | FLJ11036 |
| CD4 | C1orf21 | CORO1A | CAT | FOSL1 | FLJ11848 |
| CD47 | C1orf35 | CORO1B | CBX7 | G6PC3 | FLJ12270 |
| CD53 | C1QDC1 | COTL1 | CCNG2 | GANAB | FLJ12895 |
| CD63 | C20orf111 | CR1 | CCNI | GJA4 | FLJ13197 |
| CD74 | C20orf38 | CRN7 | CD34 | GLT25D1 | FLJ13646 |
| CD83 | C20orf42 | CSF1 | CD9 | GPR1 | FLJ14297 |
| CD84 | C21orf107 | CSF1R | CDC16 | GRB10 | FLJ20010 |
| CD86 | C6orf130 | CSGlcA-T | CDC5L | GREM1 | FLJ20265 |
| CDC14B | C9orf55 | CTSB | CDK5RAP1 | GRP58 | FLJ20315 |
| CDC25B | C9orf61 | CXCL1 | CDS1 | GTSE1 | FLJ20345 |
| CDC6 | CA11 | CXCL10 | CELSR1 | HAPLN1 | FLJ20457 |
| CDC7 | CAB39L | CXCL9 | Cep152 | HCA112 | FLJ20489 |
| CDH11 | CABC1 | CXCR4 | CES1 | HDGFRP3 | FLJ20604 |
| CEBPD | CAPN3 | CXorf9 | CFH | HEM1 | FLJ20618 |
| CECR1 | CAT | CYLD | CGI-143 | HIP1 | FLJ20701 |
| CENTA2 | CBX7 | CYP26A1 | CGI-41 | HKE2 | FLJ20859 |
| CETP | CCNG2 | DACT1 | CHCHD2 | HLA-B | FLJ20920 |
| CFLAR | CCNI | DAPK3 | CHN2 | HLA-C | FLJ21069 |
| CH25H | CD34 | DEPDC6 | CLASP1 | HLA-DPA1 | FLJ21511 |
| CHC1 | CD9 | DKFZP434B044 | CLASP2 | HLA-DRA | FLJ23091 |
| CHD7 | CDC16 | DKFZP434H132 | CLCA4 | HLA-DRB1 /// | FLJ23186 |
| CHN1 | CDC42BPA | DKFZP434P211 | CLDN1 | HLA-DRB3 /// | FLJ23861 |
| CHST1 | CDC5L | DKFZp566C0424 | CLDN8 | HLA-DRB4 | FOXC1 |
| cig5 | CDH12 | DKFZp762C186 | CNKSR1 | HLA-DRB6 | FRMD4A |
| CINP | CDK11 | DKFZp762E1312 | COBL | HNT | FXYD3 |
| CKAP1 | CDK5RAP1 | DNM3 | CORO2B | HPCAL1 | FYCO1 |
| CKIP-1 | CDS1 | DOK5 | COX15 | HRH1 | GAL3ST4 |
| CLN2 | CELSR1 | DPT | COX4I1 | HS3ST3A1 | GALNT11 |
| CMKLR1 | Cep152 | DPYSL2 | COX7C | HSGP25L2G | GATM |
| CNN2 | CES1 | DPYSL3 | CP | IGH@ /// IGHG1 | GDPD2 |
| CNTNAP1 | CFH | ECGF1 | CPE | IGLJ3 /// IGLC2 | GHITM |
| COL10A1 | CGI-143 | EFEMP2 | CRBN | IGLL1 | GLRX |
| COL11A1 | CGI-41 | EFHD2 | CRKL | ILT8 | GLTSCR2 |
| COL15A1 | CGI-51 | EMILIN1 | CRYZL1 | INHBA | GOLGA7 |
| COL1A1 | CHCHD2 | EPHA3 | CS | ISG20 | GPM6B |
| COL4A1 | CHN2 | EPHB2 | CTDSPL | KCNJ8 | GPR48 |
| COL4A2 | CHS1 | EPIM | CTNNBIP1 | KDELR3 | GPR87 |
| COL4A4 | CLASP1 | ERP70 | CTNND1 | KERA | GRHPR |
| COL5A1 | CLASP2 | FAD104 | CTSF | KIAA0040 | GSTA3 |
| COL5A2 | CLCA4 | FAP | CTSL2 | KIAA0746 | GULP1 |
| COL6A1 | CLDN1 | FARS1 | CUL3 | KIAA0792 | H2AFJ |
| COL6A3 | CLDN8 | FBN2 | CYP2J2 | KIAA1644 | HBS1L |
| COL8A1 | CNKSR1 | FCGR1A | CYP39A1 | LBH | HCNGP |
| COL8A2 | COBL | FCGR2A | CYP3A5 | LBP | HEBP1 |
| COMP | CORO2B | FGF13 | CYP4F12 | LGALS3BP | HERC2 |
| COPS8 | COX15 | FKBP11 | CYP4F3 | LILRA2 | HEY2 |
| CORO1A | COX4I1 | FLJ10292 | D2LIC | LILRB2 | HNRPD |
| CORO1B | COX7C | FLJ10385 | DAF | LMCD1 | HOXA9 |
| COTL1 | CP | FLJ10647 | DAG1 | LOC146489 | HOXC10 |
| CR1 | CPE | FLJ10815 | DBI | LOC254359 | HPS4 |
| CRN7 | CRBN | FLJ11259 | DCN | LOC284021 | HRASLS |
| CSF1 | CRKL | FLJ12438 | DCT | LOC51334 | HSD17B7 |
| CSF1R | CRYZL1 | FLJ12439 | DDR1 | LR8 | IDE |
| CSGlcA-T | CS | FLJ12443 | DDX42 | LRCH4 | IDS |
| CSPG2 | CTDSPL | FLJ13725 | DGCR2 | MAGEB4 | IHPK2 |
| CSRP2 | CTNNBIP1 | FLJ22175 | DGKA | MAP4K1 | IL1F7 |
| CTGF | CTNND1 | FLJ23221 | DHX57 | MARCKS | IL20RA |
| CTSB | CTSF | FLJ23556 | DKFZp434C0328 | MCM5 | IL22RA1 |
| CTSC | CTSL2 | FLJ30656 | DKFZp434N2030 | MDS018 | ING4 |
| CTSL | CUL3 | FLNA | DKFZp667G2110 | MGC16664 | INPP5A |
| CXCL1 | CYP2J2 | FLOT2 | DKFZp761P211 | MGC27165 /// | IRS3L |
| CXCL10 | CYP39A1 | FLRT2 | DLG3 | IGH@ /// IGHG1 | ITPR1 |
| CXCL9 | CYP3A5 | FMO3 | DNAL4 | /// IGHM | KCNJ13 |
| CXCR4 | CYP4F12 | FOLH1 | DNCH2 | MGC27165 /// | KCNK1 |
| CXorf9 | CYP4F3 | FOSL1 | DOCK9 | IGHG1 | KIAA0033 |
| CYBA | D2LIC | FTHP1 | DOK4 | MGC4294 | KIAA0495 |
| CYBB | DAF | FTL | DSG2 | MGC4368 | KIAA0794 |
| CYLD | DAG1 | FZD2 | DSTN | MICAL2 | KIAA1305 |
| CYP26A1 | DBI | G6PC3 | DTNA | MICAL-L2 | KIAA1815 |
| CYP7B1 | DCN | GAA | DVL1 | MMP19 | KIF1B |
| CYR61 | DCT | GADD45B | EBP | MS4A4A | KIT |
| D2S448 | DDB1 | GALNT10 | ECHDC3 | MS4A6A | KLHDC2 |
| DAB2 | DDR1 | GANAB | EDD | MTRF1L | LAMC3 |
| DACT1 | DDX42 | GARP | EFHC2 | MTVR1 | LASS6 |
| DAP | DF | GDF15 | EFNB2 | MXD4 | LGTN |
| DAPK3 | DGCR2 | GGTLA1 | EFS | MYO9B | LIN7B |
| DCTD | DGKA | GJA4 | EGF | NAGA | LOC254531 |
| DEPDC6 | DHX57 | GLA | EGFL5 | NCBP1 | LOC51136 |
| DIO2 | DKFZp434C0328 | GLT25D1 | EIF2B1 | NEDD9 | LOC51149 |
| DKFZP434B044 | DKFZP434C212 | GNLY | EIF2C1 | NF1 | LOC63928 |
| DKFZP434H132 | DKFZp434N2030 | GPR1 | eIF3k | NINJ2 | LOC81558 |
| DKFZP434P211 | DKFZP586H2123 | GPSM3 | EIF3S3 | NOX4 | LRP16 |
| DKFZp564I1922 | DKFZp667G2110 | GPX7 | EIF3S6IP | NPR3 | LRP1B |
| DKFZP564K0822 | DKFZp761P211 | GRB10 | EIF4B | NRG1 | LRRC16 |
| DKFZp566C0424 | DLEU1 | GREM1 | ELF5 | NRIP3 | LRRC2 |
| DKFZp762C186 | DLG3 | GRP58 | ELL3 | NRP1 | LRRFIP2 |
| DKFZp762E1312 | DNAL4 | GTSE1 | ELOVL4 | OAS3 | LUC7L |
| DNAJC7 | DNCH2 | GUCY1A3 | EMP2 | OR2B2 | MAGEF1 |
| DNM3 | DNCI1 | HA-1 | EMX2 | OSBPL10 | MCCC1 |
| DNMT1 | DOCK9 | HAPLN1 | EPB41L4B | P101-PI3K | MCCC2 |
| DOK5 | DOK4 | HCA112 | EPHB6 | P2RY13 | MCOLN3 |
| DPT | DSCR1L1 | HDGFRP3 | EPHX1 | PAPPA | MDH1 |
| DPYSL2 | DSG2 | HEG | EPHX2 | PARP8 | MFN1 |
| DPYSL3 | DSTN | HEM1 | EPN2 | PCDH12 | MGC3123 |
| ECGF1 | DTNA | HFE | ERBB3 | PDGFC | MGC5139 |
| EFEMP2 | DVL1 | HIP1 | ERCC3 | PDLIM7 | MLL |
| EFHD2 | EBP | HKE2 | ERCC5 | Pfs2 | MMP27 |
| EIF4A1 | ECHDC3 | HLA-B | ETFDH | PHTF1 | MOCS2 |
| EMILIN1 | EDD | HLA-C | EXTL2 | PI4KII | MPPE1 |
| ENG | EFHC2 | HLA-DPA1 | EZH1 | PILRA | MRPS30 |
| EPHA3 | EFNA4 | HLA-DRA | FAHD2A | PITPNC1 | MST4 |
| EPHB2 | EFNB2 | HLA-DRB1 /// | FAM8A1 | PLAU | MSTP9 |
| EPIM | EFS | HLA-DRB3 /// | FBLN1 | PLSCR3 | NALP1 |
| ERP70 | EGF | HLA-DRB4 | FBXO21 | PLVAP | NAV2 |
| FAD104 | EGFL5 | HLA-DRB6 | FBXO42 | PLXDC1 | NCOA1 |
| FAP | EIF2B1 | HNT | FBXO9 | PP2447 | NDRG2 |
| FARS1 | EIF2C1 | HPCAL1 | FDFT1 | PPM1F | NDRG3 |
| FBN2 | eIF3k | HRH1 | FGFR1 | PRO1855 | NEIL1 |
| FCER1G | EIF3S3 | HS3ST3A1 | FGFR2 | PSCD4 | NFS1 |
| FCGR1A | EIF3S6IP | HSGP25L2G | FH | PTGER4 | NIP30 |
| FCGR2A | EIF4B | HTR2A | FHL1 | RAB15 | NIT2 |
| FGF13 | ELF5 | HTR2C | FLJ10415 | RAB20 | NOL3 |
| FKBP11 | ELL3 | ICAM1 | FLJ10521 | RAB35 | NOTCH2 |
| FLJ10292 | ELOVL4 | ICAM2 | FLJ10769 | RAMP | NPY1R |
| FLJ10385 | EMP2 | IF | FLJ10847 | RCN3 | OCLN |
| FLJ10647 | EMX2 | IFI35 | FLJ10948 | RELA | ORC4L |
| FLJ10815 | EPB41L4B | IFIT2 | FLJ10986 | RFX5 | PAQR6 |
| FLJ11259 | EPHB6 | IFIT3 | FLJ11036 | RIN3 | PARD3 |
| FLJ12438 | EPHX1 | IFIT5 | FLJ11220 | RIOK3 | PAWR |
| FLJ12439 | EPHX2 | IGF1 | FLJ11848 | RNF17 | PCSK2 |
| FLJ12443 | EPN2 | IGFBP2 | FLJ12270 | RRM2 | PDCD4 |
| FLJ13725 | ERBB3 | IGH@ /// IGHG1 | FLJ12895 | SAA1 /// SAA2 | PDCD6IP |
| FLJ22175 | ERCC3 | IGHM | FLJ13197 | SCAND1 | PDGFD |
| FLJ23221 | ERCC5 | IGKV1D-13 | FLJ13646 | SEC61A1 | PDHA1 |
| FLJ23556 | ETFA | IGLC2 | FLJ13910 | SEMA6B | PECI |
| FLJ30656 | ETFDH | IGLJ3 /// IGLC2 | FLJ14297 | SH3TC1 | PELI2 |
| FLNA | EXTL2 | IGLL1 | FLJ20010 | SHOX2 | PERLD1 |
| FLOT1 | EZH1 | IL10RA | FLJ20265 | SLAMF8 | PGRMC1 |
| FLOT2 | F10 | IL2RG | FLJ20315 | SLC11A1 | PGRMC2 |
| FLRT2 | FAHD2A | IL4R | FLJ20345 | SLC12A8 | PJA1 |
| FMO3 | FAM8A1 | IL6 | FLJ20457 | SLC15A3 | PLP1 |
| FN1 | FBLN1 | ILT8 | FLJ20489 | SLC16A1 | PLXNA3 |
| FOLH1 | FBXO21 | INHBA | FLJ20604 | SLC24A6 | POGK |
| FOLR2 | FBXO28 | IRF1 | FLJ20618 | SLC25A22 | POLR3E |
| FOSL1 | FBXO42 | IRF7 | FLJ20701 | SLC2A14 /// | POU2F3 |
| FTH1 | FBXO9 | ISG20 | FLJ20859 | SLC2A3 | PPA2 |
| FTHP1 | FDFT1 | ITGA5 | FLJ20920 | SLC2A3 | PPP1CB |
| FTL | FGFR1 | ITGAM | FLJ21069 | SLC43A3 | PPT2 |
| FZD2 | FGFR2 | ITGAX | FLJ21511 | SLC4A7 | PREP |
| G1P2 | FH | ITGBL1 | FLJ23091 | SLCO1A2 | PSAT1 |
| G1P3 | FHL1 | ITIH3 | FLJ23186 | SN | PTD015 |
| G6PC3 | FLJ10326 | K-ALPHA-1 | FLJ23861 | SNFT | PWP1 |
| GAA | FLJ10415 | KCNJ15 | FLJ30092 | SNX11 | RAB33B |
| GADD45B | FLJ10521 | KCNJ8 | FLNB | SOD2 | RAB3-GAP150 |
| GALNT10 | FLJ10769 | KCNQ1 | FMO5 | SPHK1 | RABL2A /// |
| GANAB | FLJ10847 | KDELR3 | FNBP2 | SPHK2 | RABL2B |
| GARP | FLJ10948 | KERA | FOXC1 | SPTLC2 | RAD54B |
| GBP1 | FLJ10986 | KIAA0040 | FOXJ3 | SSH1 | RAI2 |
| GDF15 | FLJ11036 | KIAA0342 | FRMD4A | STC2 | REC14 |
| GEM | FLJ11220 | KIAA0508 | FRZB | SYNJ2 | RGNEF |
| GGTLA1 | FLJ11848 | KIAA0602 | FXYD3 | TACC3 | RHOT1 |
| GJA4 | FLJ12270 | KIAA0746 | FYCO1 | TAL1 | RNF128 |
| GLA | FLJ12895 | KIAA0792 | FZD7 | TAZ | RPL13 |
| GLT25D1 | FLJ13197 | KIAA0894 | GABBR1 | TBC1D5 | RPL13A |
| GMFG | FLJ13646 | KIAA0963 | GAL3ST4 | TBX2 | RPL17 |
| GNG5 | FLJ13910 | KIAA1036 | GALNT11 | TMEFF1 | RPL29 |
| GNLY | FLJ14297 | KIAA1644 | GALNT3 | TMEM2 | RPL3 |
| GPR1 | FLJ20010 | LAIR1 | GATA3 | TMEM8 | RPL31 |
| GPSM3 | FLJ20265 | LAPTM5 | GATM | TMPRSS4 | RPL7A |
| GPX1 | FLJ20315 | LBH | GCHFR | TNFAIP2 | RRAGD |
| GPX7 | FLJ20345 | LBP | GDPD2 | TNFRSF12A | RTN3 |
| GRB10 | FLJ20457 | LGALS1 | GGPS1 | TNFRSF21 | RYK |
| GREM1 | FLJ20489 | LGALS3BP | GHITM | TNFRSF6B | SARG |
| GRP58 | FLJ20604 | LGALS9 | GLRX | TNFSF12- | SCAMP1 |
| GTSE1 | FLJ20618 | LILRA2 | GLS2 | TNFSF13 /// | SERPINB13 |
| GUCY1A3 | FLJ20701 | LILRB2 | GLTSCR2 | TNFSF13 | SERTAD3 |
| HA-1 | FLJ20859 | LMCD1 | GNPAT | TNIP2 | SETMAR |
| HAPLN1 | FLJ20920 | LOC146489 | GNRH1 | TNRC5 | SFRS11 |
| HCA112 | FLJ21069 | LOC254359 | GOLGA7 | TPMT | SGPL1 |
| HCK | FLJ21511 | LOC284021 | GPLD1 | TRAF3 | SLC19A2 |
| HCLS1 | FLJ23091 | LOC51334 | GPM6A | TREM2 | SLC25A12 |
| HDGFRP3 | FLJ23186 | LOC56902 | GPM6B | TRIAD3 | SLC35F2 |
| HEG | FLJ23861 | LR8 | GPNMB | TXNDC5 | SLC6A14 |
| HEM1 | FLJ30092 | LRCH4 | GPR48 | UBE2I | SNCA |
| HFE | FLNB | LTB | GPR56 | WAS | SP192 |
| HIP1 | FMO5 | LY64 | GPR87 | WASPIP | SPATS2 |
| HKE2 | FNBP2 | LY6E | GPRASP1 | YWHAH | SSB3 |
| HLA-B | FOXC1 | LY86 | GREB1 | ZC3HDC1 | SSH3 |
| HLA-C | FOXJ3 | M6PRBP1 | GRHPR | ZDHHC14 | ST13 |
| HLA-DMA | FRMD4A | MAGEB4 | GSTA3 | ZFP36L2 | STS |
| HLA-DMB | FRZB | MAN2B1 | GSTA4 | | STXBP6 |
| HLA-DPA1 | FXYD3 | MAP4K1 | GSTT1 | | SVIL |
| HLA-DRA | FYCO1 | MARCKS | GULP1 | | TAF7L |
| HLA-DRB1 /// | FZD7 | MATN3 | H2AFJ | | TDE1 |
| HLA-DRB3 /// | GABARAPL2 | MCM5 | HARSL | | TFAP2A |
| HLA-DRB4 | GABBR1 | MDS018 | HBP1 | | TFAP2B |
| HLA-DRB3 | GAL3ST4 | METTL1 | HBS1L | | TFB1M |
| HLA-DRB6 | GALNT11 | MFAP2 | HCNGP | | TM4SF6 |
| HNT | GALNT3 | MFNG | HEBP1 | | TMOD1 |
| HPCAL1 | GATA3 | MGC16664 | HERC2 | | TNMD |
| HRH1 | GATM | MGC27165 /// | HEY2 | | TPD52L1 |
| HS3ST3A1 | GCHFR | IGH@ /// IGHG1 | HLF | | TRIM2 |
| HSGP25L2G | GDPD2 | /// IGHM | HMGCS2 | | TRIT1 |
| HSPG2 | GGPS1 | MGC27165 /// | HNRPD | | TUFT1 |
| HTR2A | GHITM | IGHG1 | HOP | | TULP4 |
| HTR2C | GLCE | MGC3047 | HOXA9 | | TXNDC4 |
| ICAM1 | GLRB | MGC4294 | HOXC10 | | UBAP2 |
| ICAM2 | GLRX | MGC4368 | HOXD4 | | Ufc1 |
| IF | GLS2 | MICAL2 | HPGD | | USP21 |
| IFI16 | GLTSCR2 | MICAL-L2 | HPS4 | | USP34 |
| IFI27 | GNPAT | MICB | HRASLS | | VAV3 |
| IFI30 | GNRH1 | MMP11 | HSD11B1 | | XLKD1 |
| IFI35 | GOLGA7 | MMP14 | HSD17B7 | | XPNPEP1 |
| IFI44 | GPLD1 | MMP19 | HSPB3 | | ZDHHC11 |
| IFIT2 | GPM6A | MMP3 | IDE | | ZDHHC13 |
| IFIT3 | GPM6B | MMP9 | IDS | | ZFD25 |
| IFIT5 | GPNMB | MOXD1 | IGBP1 | | ZFP161 |
| IFITM1 | GPR48 | MS4A4A | IGFBP5 | | ZFYVE21 |
| IFITM2 | GPR56 | MS4A6A | IHPK2 | | ZNF16 |
| IFNGR2 | GPR87 | MT1F | IL11RA | | ZNF224 |
| IGF1 | GPRASP1 | MTRF1L | IL1F7 | | ZNF266 |
| IGF2 | GREB1 | MTVR1 | IL20RA | | ZNF395 |
| IGFBP2 | GRHPR | MVP | IL22RA1 | | ZNF506 |
| IGFBP3 | GSN | MXD4 | ING4 | | |
| IGFBP4 | GSTA3 | MYO9B | INPP5A | | |
| IGFBP7 | GSTA4 | NAGA | IRS3L | | |
| IGH@ /// IGHG1 | GSTM5 | NCBP1 | IRX5 | | |
| IGHG1 | GSTT1 | NEDD9 | ITPR1 | | |
| IGHM | GTF2I /// | NF1 | ITPR2 | | |
| IGKC | GTF2IP1 | NINJ2 | JARID1B | | |
| IGKV1D-13 | GULP1 | NK4 | JMJD1B | | |
| IGLC2 | H2AFJ | NKG7 | KAZALD1 | | |
| IGLJ3 /// IGLC2 | HARSL | NOX4 | KCNJ13 | | |
| IGLL1 | HBP1 | NPR3 | KCNK1 | | |
| IL10RA | HBS1L | NR2F1 | KIAA0033 | | |
| IL2RG | HCNGP | NRG1 | KIAA0157 | | |
| IL4R | HEBP1 | NRGN | KIAA0174 | | |
| IL6 | HERC2 | NRIP3 | KIAA0182 | | |
| ILT8 | HEY2 | NRP1 | KIAA0232 | | |
| INHBA | HLF | NRP2 | KIAA0240 | | |
| IRF1 | HMGCR | NUDT3 | KIAA0265 | | |
| IRF7 | HMGCS2 | OAS1 | KIAA0368 | | |
| ISG20 | HNRPD | OAS3 | KIAA0459 | | |
| ITGA5 | HOP | OLFML2B | KIAA0495 | | |
| ITGAM | HOXA9 | OR2B2 | KIAA0515 | | |
| ITGAX | HOXC10 | OSBPL10 | KIAA0553 | | |
| ITGB2 | HOXD4 | P101-PI3K | KIAA0582 | | |
| ITGBL1 | HPGD | P2RX4 | KIAA0657 | | |
| ITIH3 | HPS4 | P2RY13 | KIAA0703 | | |
| K-ALPHA-1 | HRASLS | PAFAH1B2 | KIAA0794 | | |
| KCNJ15 | HSD11B1 | PAPPA | KIAA0828 | | |
| KCNJ8 | HSD17B4 | PAPSS2 | KIAA0895 | | |
| KCNQ1 | HSD17B7 | PARP8 | KIAA1007 | | |
| KDELR3 | HSPB3 | PARVB | KIAA1018 | | |
| KERA | IDE | PCDH12 | KIAA1093 | | |
| KIAA0040 | IDS | PCDH17 | KIAA1117 | | |
| KIAA0101 | IGBP1 | PCSK5 | KIAA1155 | | |
| KIAA0342 | IGFBP5 | PDE1A | KIAA1305 | | |
| KIAA0508 | IGFBP6 | PDGFC | KIAA1467 | | |
| KIAA0602 | IHPK2 | PDLIM7 | KIAA1536 | | |
| KIAA0746 | IL11RA | PDXK | KIAA1815 | | |
| KIAA0792 | IL1F7 | PENK | KIF1B | | |
| KIAA0894 | IL20RA | Pfs2 | KIT | | |
| KIAA0963 | IL22RA1 | PGM3 | KLHDC2 | | |
| KIAA0992 | ING4 | PHTF1 | KLK1 | | |
| KIAA1036 | INPP5A | PI4KII | KRT15 | | |
| KIAA1199 | INSIG2 | PILRA | L3MBTL | | |
| KIAA1462 | IRS3L | PITPNC1 | LAMC3 | | |
| KIAA1644 | IRX5 | PKN1 | LANCL1 | | |
| LAIR1 | ITPR1 | PLAU | LASS6 | | |
| LAPTM5 | ITPR2 | PLCB2 | LEPR | | |
| LBH | JARID1B | PLEK | LETMD1 | | |
| LBP | JMJD1B | PLSCR3 | LGTN | | |
| LGALS1 | KAZALD1 | PLTP | LIN7B | | |
| LGALS3BP | KCNJ13 | PLVAP | LOC114977 | | |
| LGALS9 | KCNK1 | PLXDC1 | LOC157567 | | |
| LGMN | KIAA0033 | PML | LOC201725 /// TOMM7 | | |
| LHFPL2 | KIAA0157 | POLD1 | LOC254531 | | |
| LILRA2 | KIAA0174 | POLE2 | LOC51136 | | |
| LILRB1 /// LYZ | KIAA0182 | POSTN | LOC51149 | | |
| LILRB2 | KIAA0232 | PP2447 | LOC63928 | | |
| LIPA | KIAA0240 | pp9099 | LOC81558 | | |
| LMCD1 | KIAA0251 | PPIB | LRP16 | | |
| LMNB1 | KIAA0265 | PPM1F | LRP1B | | |
| LNK | KIAA0368 | PRELP | LRRC16 | | |
| LOC146489 | KIAA0459 | PRKR | LRRC2 | | |
| LOC254359 | KIAA0494 | PRO1855 | LRRFIP2 | | |
| LOC284021 | KIAA0495 | PSCD4 | LSS | | |
| LOC51334 | KIAA0515 | PSMB10 | LTA4H | | |
| LOC56902 | KIAA0553 | PTGER4 | LUC7L | | |
| LOXL1 | KIAA0582 | PTP4A2 | MAGEA11 | | |
| LOXL2 | KIAA0657 | PTPN18 | MAGEF1 | | |
| LR8 | KIAA0703 | RAB13 | MAN2A2 | | |
| LRCH4 | KIAA0794 | RAB15 | MAP3K4 | | |
| LTB | KIAA0828 | RAB20 | MASK-BP3 /// EIF4EBP3 | | |
| LTBP2 | KIAA0895 | RAB35 | MATN2 | | |
| LTF | KIAA1007 | RAC2 | MBP | | |
| LUM | KIAA1018 | RAMP | MCCC1 | | |
| LY64 | KIAA1093 | RASGRP2 | MCCC2 | | |
| LY6E | KIAA1117 | RCN3 | MCOLN3 | | |
| LY86 | KIAA1155 | RELA | MDH1 | | |
| LY96 | KIAA1305 | RELB | METTL3 | | |
| LYN | KIAA1467 | RFX5 | MFN1 | | |
| M6PRBP1 | KIAA1536 | RGS16 | MGC22014 | | |
| MAGEB4 | KIAA1815 | RIN3 | MGC2650 | | |
| MAN2B1 | KIF1B | RIOK3 | MGC3123 | | |
| MAP4K1 | KIT | RNF17 | MGC48332 | | |
| MAP4K4 | KLHDC2 | RODH | MGC5139 | | |
| MAPRE1 | KLK1 | RRM2 | MGEA5 | | |
| MARCKS | KRT15 | RUNX1 | MLL | | |
| MATN3 | L3MBTL | SAA1 /// SAA2 | MMP27 | | |
| MCM5 | LAMC3 | SAMHD1 | MOAP1 | | |
| MDK | LANCL1 | SCAND1 | MOCS2 | | |
| MDS018 | LASS6 | SCG2 | MPPE1 | | |
| METTL1 | LEPR | SCO2 | MRPS30 | | |
| MFAP2 | LETMD1 | SDC3 | MRS2L | | |
| MFNG | LGTN | SEC61A1 | MSH5 | | |
| MGAT1 | LIN7B | SECTM1 | MST1 | | |
| MGC16664 | LKAP | SELL | MST4 | | |
| MGC27165 | LOC114977 | SEMA6B | MSTP9 | | |
| MGC27165 /// | LOC157567 | SGCD | MTCP1 | | |
| IGH@ /// IGHG1 | LOC201725 /// | SH3TC1 | MTMR3 | | |
| /// IGHM | TOMM7 | SHOX2 | MTMR4 | | |
| MGC27165 /// | LOC254531 | SIL | MUT | | |
| IGHG1 | LOC51136 | SIPA1 | MXI1 | | |
| MGC3047 | LOC51149 | SLAMF8 | NAB1 | | |
| MGC4294 | LOC63928 | SLC11A1 | NAB2 | | |
| MGC4368 | LOC81558 | SLC12A8 | NACA | | |
| MICAL2 | LONP | SLC15A3 | NAG | | |
| MICAL-L2 | LRBA | SLC16A1 | NALP1 | | |
| MICB | LRP16 | SLC1A3 | NAV2 | | |
| MMP11 | LRP1B | SLC20A1 | NAV3 | | |
| MMP14 | LRRC16 | SLC24A6 | NCOA1 | | |
| MMP19 | LRRC2 | SLC25A22 | NDRG2 | | |
| MMP3 | LRRFIP2 | SLC2A14 /// | NDRG3 | | |
| MMP9 | LSS | SLC2A3 | NDUFB6 | | |
| MOXD1 | LTA4H | SLC2A3 | NDUFC1 | | |
| MRPS6 | LUC7L | SLC39A14 | NEBL | | |
| MS4A4A | MAGEA11 | SLC43A3 | NEIL1 | | |
| MS4A6A | MAGEF1 | SLC4A7 | NFATC3 | | |
| MSN | MAN2A2 | SLC7A7 | NFIB | | |
| MT1F | MAP3K4 | SLCO1A2 | NFS1 | | |
| MTHFD2 | MASK-BP3 /// | SMA4 | NFYC | | |
| MTRF1L | EIF4EBP3 | SN | NIP30 | | |
| MTVR1 | MATN2 | SNFT | NIT2 | | |
| MVP | MATN4 | SNX11 | NOL3 | | |
| MX1 | MBP | SOD2 | NOTCH2 | | |
| MX2 | MCCC1 | SPHK1 | NPAS1 | | |
| MXD4 | MCCC2 | SPHK2 | NPR2 | | |
| MYD88 | MCOLN3 | SPTLC2 | NPY1R | | |
| MYO5A | MDH1 | SSH1 | NR3C2 | | |
| MYO9B | MECP2 | STARD8 | NRD1 | | |
| NAGA | METTL3 | STAT2 | NUMA1 | | |
| NCBP1 | MFN1 | STC2 | OACT2 | | |
| NCF4 | MGC22014 | STMN1 | OCLN | | |
| NEDD9 | MGC2650 | STRN | ODAG | | |
| NF1 | MGC3123 | SYNJ2 | OGT | | |
| NID2 | MGC48332 | SYT11 | OLFML1 | | |
| NINJ2 | MGC5139 | T1A-2 | OLFML2A | | |
| NK4 | MGEA5 | TACC3 | ORC4L | | |
| NKG7 | MLL | TAL1 | OSBPL1A | | |
| NMI | MMP27 | TAP2 | OSBPL2 | | |
| NNMT | MOAP1 | TAZ | OSR2 | | |
| NOX4 | MOCS2 | TBC1D5 | P8 | | |
| NPR3 | MPPE1 | TBX2 | PABPN1 | | |
| NR2F1 | MRPS30 | TBXAS1 | PAM | | |
| NRG1 | MRS2L | TGFB1I1 | PAQR6 | | |
| NRGN | MSH5 | TIE | PARD3 | | |
| NRIP3 | MST1 | TIMELESS | PAWR | | |
| NRP1 | MST4 | TK2 | PBP | | |
| NRP2 | MSTP9 | TMEFF1 | PCCA | | |
| NUDT3 | MTCP1 | TMEM2 | PCDH21 | | |
| OAS1 | MTMR1 | TMEM8 | PCSK2 | | |
| OAS2 | MTMR3 | TMPRSS4 | PDCD4 | | |
| OAS3 | MTMR4 | TMSB10 | PDCD6IP | | |
| OLFML2B | MUT | TNFAIP2 | PDGFD | | |
| OR2B2 | MXI1 | TNFRSF12A | PDGFRL | | |
| OSBPL10 | NAB1 | TNFRSF21 | PDHA1 | | |
| OSMR | NAB2 | TNFRSF4 | PDZK3 | | |
| P101-PI3K | NACA | TNFRSF6B | PECI | | |
| P2RX4 | NAG | TNSF12- | PELI2 | | |
| P2RY13 | NALP1 | TNFSF13 /// | PERLD1 | | |
| PAFAH1B2 | NAV2 | TNFSF13 | PEX1 | | |
| PAPPA | NAV3 | TNFSF4 | PFAAP5 | | |
| PAPSS2 | NCALD | TNIP2 | PFDN5 | | |
| PARP8 | NCAM1 | TNRC5 | PFKM | | |
| PARVB | NCOA1 | TOSO | PGAP1 | | |
| PCDH12 | NDRG2 | TP53I3 | PGRMC1 | | |
| PCDH17 | NDRG3 | TPMT | PGRMC2 | | |
| PCSK5 | NDUFB6 | TRAF3 | PHKA2 | | |
| PDE1A | NDUFC1 | TREM2 | PHYH | | |
| PDGFA | NDUFS4 | TRIAD3 | PIAS3 | | |
| PDGFC | NEBL | TRIO | PIGC | | |
| PDLIM1 | NEDD8 | TRIP13 | PIGH | | |
| PDLIM7 | NEIL1 | TXNDC5 | PIK3C2G | | |
| PDXK | NEK9 | TYMS | PINK1 | | |
| PECAM1 | NFATC3 | U2AF1 | PJA1 | | |
| PENK | NFIB | UBD | PLA2G4A | | |
| Pfs2 | NFS1 | UBE2I | PLCB4 | | |
| PGM3 | NFYC | UCK2 | PLEKHE1 | | |
| PHC2 | NIP30 | UPP1 | PLP1 | | |
| PHTF1 | NISCH | WAS | PLXNA3 | | |
| PI4KII | NIT2 | WASPIP | PMM1 | | |
| PILRA | NOL3 | WISP1 | POGK | | |
| PITPNC1 | NOTCH2 | YWHAH | POLR3E | | |
| PKN1 | NPAS1 | ZC3HDC1 | POU2F3 | | |
| PLAU | NPR2 | ZDHHC14 | POU6F1 | | |
| PLAUR | NPY1R | ZFP36L2 | PPA2 | | |
| PLCB2 | NR3C2 | ZNF364 | PPL | | |
| PLEK | NRD1 | RABGAP1L | PPM1A | | |
| PLSCR1 | NUMA1 | RABL2A /// RABL2B | PPOX | | |
| PLSCR3 | NUP133 | RABL3 | PPP1CB | | |
| PLTP | NY-REN-7 | RAD54B | PPP1CC | | |
| PLVAP | OACT2 | RAI2 | PPP1R7 | | |
| PLXDC1 | OCLN | RALGPS1 | PPT2 | | |
| PLXND1 | ODAG | RAPGEF3 | PRDM2 | | |
| PML | OGT | RAPGEF4 | PREP | | |
| POLD1 | OLFML1 | RBM5 | PRKCH | | |
| POLE2 | OLFML2A | RDH11 | PRPF8 | | |
| POSTN | ORC4L | REC14 | PRPSAP2 | | |
| PP2447 | OSBPL1A | RFP2 | PSAT1 | | |
| pp9099 | OSBPL2 | RGNEF | PSMD5 | | |
| PPIB | OSR2 | RHOT1 | PTD015 | | |
| PPM1F | P11 | RNASE4 | PTPN21 | | |
| PPT1 | P8 | RNF103 | PTPN3 | | |
| PRCP | PABPN1 | RNF128 | PTPRF | | |
| PRDX4 | PAM | RNF3 | PUM2 | | |
| PRELP | PAQR6 | RNF5 | PWP1 | | |
| PRG1 | PARD3 | RPL10A | QPCT | | |
| PRKR | PAWR | RPL12 | RAB11A | | |
| PRO1855 | PBP | RPL13 | RAB33B | | |
| PRPS1 | PCCA | RPL13A | RAB3-GAP150 | | |
| PRSS23 | PCDH21 | RPL15 | | | |
| PSCD4 | PCSK2 | RPL17 | | | |
| PSMB10 | PDCD4 | RPL18 | | | |
| PSMB8 | PDCD6IP | RPL22 | | | |
| PSME2 | PDGFD | RPL27 | | | |
| PTGER4 | PDGFRL | RPL29 | | | |
| PTP4A2 | PDHA1 | RPL3 | | | |
| PTPN18 | PDZK3 | RPL31 | | | |
| PTTG1IP | PECI | RPL7 | | | |
| RAB13 | PELI2 | RPL7A | | | |
| RAB15 | PERLD1 | RPL9 | | | |
| RAB20 | PEX1 | RPS20 | | | |
| RAB31 | PFAAP5 | RPS3A | | | |
| RAB35 | PFDN5 | RPS8 | | | |
| RAC2 | PFKM | RRAGD | | | |
| RAFTLIN | PGAP1 | RRM1 | | | |
| RAMP | PGRMC1 | RTN3 | | | |
| RAMP3 | PGRMC2 | RXRA | | | |
| RASGRP2 | PHKA2 | RYK | | | |
| RCN3 | PHYH | RYR3 | | | |
| RELA | PIAS3 | SAP18 | | | |
| RELB | PIGC | SARG | | | |
| REPS2 | PIGH | SCAMP1 | | | |
| RFX5 | PIK3C2G | SCEL | | | |
| RGL1 | PINK1 | SCNN1B | | | |
| RGS16 | PJA1 | SELENBP1 | | | |
| RGS3 | PLA2G4A | SERPINB13 | | | |
| RIN3 | PLCB4 | SERPINB2 | | | |
| RIOK3 | PLEKHE1 | SERTAD3 | | | |
| RNF17 | PLP1 | SETMAR | | | |
| RODH | PLXNA3 | SFRS11 | | | |
| RPS6KA2 | PMM1 | SGCA | | | |
| RRBP1 | POGK | SGPL1 | | | |
| RRM2 | POLR3E | SH2D1A | | | |
| RUNX1 | POU2F3 | SHANK2 | | | |
| SAA1 /// SAA2 | POU6F1 | SIAT10 | | | |
| SAMHD1 | PPA2 | SIAT7B | | | |
| SCAND1 | PPAP2B | SKP1A | | | |
| SCG2 | PPL | SLC14A1 | | | |
| SCO2 | PPM1A | SLC19A2 | | | |
| SDC3 | PPOX | SLC23A2 | | | |
| SEC61A1 | PPP1CB | SLC25A12 | | | |
| SECTM1 | PPP1CC | SLC25A4 | | | |
| SELL | PPP1R7 | SLC35F2 | | | |
| SELP | PPP6C | SLC6A14 | | | |
| SEMA6B | PPT2 | SLIT3 | | | |
| SERPINE1 | PRDM2 | SMARCC2 | | | |
| SERPINE2 | PREP | SNCA | | | |
| SERPINF1 | PRKCH | SNX27 | | | |
| SFRP4 | PRPF8 | SOD3 | | | |
| SGCD | PRPSAP2 | SOSTDC1 | | | |
| SH3TC1 | PSAT1 | SOX9 | | | |
| SHC1 | PSMD5 | SP192 | | | |
| SHOX2 | PTD015 | SPAG1 | | | |
| SIL | PTPN21 | SPATS2 | | | |
| SIPA1 | PTPN3 | SPTAN1 | | | |
| SLAMF8 | PTPRF | SPTBN1 | | | |
| SLC11A1 | PUM2 | SS18L1 | | | |
| SLC12A8 | PURA | SSB3 | | | |
| SLC15A3 | PWP1 | SSH3 | | | |
| SLC16A1 | QPCT | SSPN | | | |
| SLC16A3 | RAB11A | ST13 | | | |
| SLC1A3 | RAB33B | STAR | | | |
| SLC20A1 | RAB3-GAP150 | STAT6 | | | |
| SLC24A6 | RABGAP1L | STS | | | |
| SLC25A22 | RABL2A /// RABL2B | STXBP6 | | | |
| SLC2A14 /// | RABL3 | SVIL | | | |
| SLC2A3 | RAD54B | TACC2 | | | |
| SLC2A3 | RAF1 | TACSTD2 | | | |
| SLC39A14 | RAI2 | TAF7L | | | |
| SLC43A3 | RALGPS1 | TBC1D4 | | | |
| SLC4A7 | RAPGEF3 | TBC1D8 | | | |
| SLC7A7 | RAPGEF4 | TDE1 | | | |
| SLCO1A2 | RBM5 | TDRD3 | | | |
| SLCO2B1 | RBMX | TFAP2A | | | |
| SMA4 | RDH11 | TFAP2B | | | |
| SN | REC14 | TFB1M | | | |
| SNFT | RFP2 | TGFA | | | |
| SNX11 | RGNEF | THOC1 | | | |
| SOD2 | RHOT1 | TIAF1 | | | |
| SP110 | RNASE4 | TIAM1 | | | |
| SPARC | RNF103 | TM4SF3 | | | |
| SPHK1 | RNF128 | TM4SF6 | | | |
| SPHK2 | RNF3 | TMOD1 | | | |
| SPON1 | RNF5 | TNKS | | | |
| SPTLC2 | RNMT | TNMD | | | |
| SRM | RPL10A | TNNC2 | | | |
| SSH1 | RPL12 | TNXB | | | |
| STAB1 | RPL13 | TOB1 | | | |
| STARD8 | RPL13A | TPD52L1 | | | |
| STAT1 | RPL15 | TRIM2 | | | |
| STAT2 | RPL17 | TRIT1 | | | |
| STAT3 | RPL18 | TRRAP | | | |
| STC2 | RPL22 | TSC1 | | | |
| STMN1 | RPL27 | TTC15 | | | |
| STMN2 | RPL29 | TUFT1 | | | |
| STOM | RPL3 | TULP3 | | | |
| STRN | RPL31 | TULP4 | | | |
| SULF1 | RPL7 | TUSC4 | | | |
| SYNJ2 | RPL7A | TXNDC4 | | | |
| SYT11 | RPL9 | TYR | | | |
| T1A-2 | RPS20 | UBAP2 | | | |
| TACC3 | RPS3A | UBE4A | | | |
| TAGLN | RPS8 | UBE4B | | | |
| TAL1 | RRAGD | Ufc1 | | | |
| TAP2 | RRM1 | UNC13B | | | |
| TAX1BP3 | RTN3 | UREB1 | | | |
| TAZ | RXRA | USP21 | | | |
| TBC1D1 | RYK | USP34 | | | |
| TBC1D5 | RYR3 | USP46 | | | |
| TBX2 | SAP18 | USP6NL | | | |
| TBXAS1 | SARG | UST | | | |
| TCTEL1 | SCAMP1 | VAV3 | | | |
| TGFB1I1 | SCEL | VIPR1 | | | |
| TGFB3 | SCNN1B | VPS11 | | | |
| TGM2 | SELENBP1 | VPS13D | | | |
| THBS1 | SEMA3B | VPS45A | | | |
| THY1 | SERPINB13 | WASF1 | | | |
| TIE | SERPINB2 | WASF3 | | | |
| TIMELESS | SERTAD3 | WDR42A | | | |
| TIMP1 | SETMAR | WISP2 | | | |
| TK2 | SFRS11 | XK | | | |
| TLR2 | SGCA | XLKD1 | | | |
| TMEFF1 | SGCG | XPNPEP1 | | | |
| TMEM2 | SGPL1 | XPO7 | | | |
| TMEM8 | SH2D1A | ZDHHC11 | | | |
| TMPRSS4 | SHANK2 | ZDHHC13 | | | |
| TMSB10 | SIAT10 | ZDHHC3 | | | |
| TNC | SIAT7B | ZFD25 | | | |
| TNFAIP2 | SKP1A | ZFP161 | | | |
| TNFRSF10B | SLC14A1 | ZFYVE21 | | | |
| TNFRSF12A | SLC19A2 | ZFYVE26 | | | |
| TNFRSF1B | SLC23A2 | ZNF10 | | | |
| TNFRSF21 | SLC25A12 | ZNF133 | | | |
| TNFRSF4 | SLC25A3 | ZNF16 | | | |
| TNFRSF6B | SLC25A4 | ZNF175 | | | |
| TNFSF12- | SLC35A1 | ZNF204 | | | |
| TNFSF13 /// | SLC35F2 | ZNF211 | | | |
| TNFSF13 | SLC6A14 | ZNF224 | | | |
| TNFSF4 | SLC9A6 | ZNF263 | | | |
| TNIP2 | SLIT3 | ZNF266 | | | |
| TNRC5 | SMARCC2 | ZNF395 | | | |
| TOSO | SNCA | ZNF506 | | | |
| TP53I3 | SNX27 | ZNF516 | | | |
| TPMT | SOD3 | ZNF539 | | | |
| TPST1 | SOSTDC1 | ZNF6 | | | |
| TRAF3 | SOX9 | ZNF647 | | | |
| TREM2 | SP192 | ZNF75 | | | |
| TRIAD3 | SPAG1 | ZNF91 | | | |
| TRIB2 | SPATS2 | | | | |
| TRIM22 | SPINK5 | | | | |
| TRIO | SPTAN1 | | | | |
| TRIP13 | SPTBN1 | | | | |
| TXNDC5 | SS18L1 | | | | |
| TYMS | SSB3 | | | | |
| TYROBP | SSBP2 | | | | |
| U2AF1 | SSH3 | | | | |
| UBD | SSPN | | | | |
| UBE2I | ST13 | | | | |
| UBE2L6 | STAR | | | | |
| UBE2S | STAT6 | | | | |
| UCK2 | STS | | | | |
| UCP2 | STXBP6 | | | | |
| UPP1 | SVIL | | | | |
| VAMP5 | TACC2 | | | | |
| VCAM1 | TACSTD2 | | | | |
| VSIG4 | TAF7L | | | | |
| VWF | TBC1D4 | | | | |
| WARS | TBC1D8 | | | | |
| WAS | TCFL5 | | | | |
| WASPIP | TDE1 | | | | |
| WISP1 | TDRD3 | | | | |
| YWHAH | TFAP2A | | | | |
| ZC3HDC1 | TFAP2B | | | | |
| ZDHHC14 | TFB1M | | | | |
| ZFP36L2 | TGFA | | | | |
| ZNF364 | THOC1 | | | | |
| ZYX | THRAP1 | | | | |
| | TIAF1 | | | | |
| | TIAM1 | | | | |
| | TM4SF11 | | | | |
| | TM4SF3 | | | | |
| | TM4SF6 | | | | |
| | TMOD1 | | | | |
| | TNA | | | | |
| | TNKS | | | | |
| | TNMD | | | | |
| | TNNC2 | | | | |
| | TNNI2 | | | | |
| | TNXB | | | | |
| | TOB1 | | | | |
| | TPD52L1 | | | | |
| | TRIM2 | | | | |
| | TRIT1 | | | | |
| | TRRAP | | | | |
| | TSC1 | | | | |
| | TTC15 | | | | |
| | TUFT1 | | | | |
| | TULP3 | | | | |
| | TULP4 | | | | |
| | TUSC4 | | | | |
| | TXNDC4 | | | | |
| | TYR | | | | |
| | UBAP2 | | | | |
| | UBE4A | | | | |
| | UBE4B | | | | |
| | Ufc1 | | | | |
| | UNC13B | | | | |
| | UQCRC2 | | | | |
| | UREB1 | | | | |
| | USP21 | | | | |
| | USP34 | | | | |
| | USP46 | | | | |
| | USP6NL | | | | |
| | UST | | | | |
| | VAV3 | | | | |
| | VIPR1 | | | | |
| | VPS11 | | | | |
| | VPS13D | | | | |
| | VPS45A | | | | |
| | WASF1 | | | | |
| | WASF3 | | | | |
| | WDR42A | | | | |
| | WIF1 | | | | |
| | WISP2 | | | | |
| | XK | | | | |
| | XLKD1 | | | | |
| | XPNPEP1 | | | | |
| | XPO7 | | | | |
| | ZDHHC11 | | | | |
| | ZDHHC13 | | | | |
| | ZDHHC3 | | | | |
| | ZFD25 | | | | |
| | ZFP161 | | | | |
| | ZFYVE21 | | | | |
| | ZFYVE26 | | | | |
| | ZNF10 | | | | |
| | ZNF133 | | | | |
| | ZNF16 | | | | |
| | ZNF175 | | | | |
| | ZNF204 | | | | |
| | ZNF211 | | | | |
| | ZNF224 | | | | |
| | ZNF263 | | | | |
| | ZNF266 | | | | |
| | ZNF273 | | | | |
| | ZNF395 | | | | |
| | ZNF506 | | | | |
| | ZNF516 | | | | |
| | ZNF539 | | | | |
| | ZNF6 | | | | |
| | ZNF647 | | | | |
| | ZNF75 | | | | |
| | ZNF91 | | | | |

This study has demonstrated that multi-center studies of skin gene profiles in scleroderma can generate meaningful results not confounded by the source of material, and show that the scleroderma transcript profile is very robust. We identified changes in matrix expression, such as a disproportionate increase in expression of collagen XI, consistent with an invocation of a fibrocartilage program in the fibrotic process. One scleroderma patient sample, notably younger (29 years) than the others, was an outlier. Thus, gene profiling can also be used to distinguish between different subtypes of scleroderma.

Other embodiments are within the scope of the following claims.

What is claimed is:

1. A method of treating or preventing scleroderma or other fibrotic disorder, the method comprising:
administering, to a subject, a Wnt signalling antagonist, in an amount effective to treat or prevent scleroderma.

2. The method of claim 1 wherein the Wnt signalling antagonist is an antagonist of canonical Wnt signalling.

3. The method of claim 1 wherein the Wnt signalling antagonist is an antagonist of non-canonical Wnt signalling.

4. The method of claim 1 wherein the Wnt signalling antagonist comprises a protein.

5. The method of claim 1 wherein the Wnt signalling antagonist is a Wnt binding protein.

6. The method of claim 5 wherein the Wnt signalling antagonist comprises a protein at least 90% identical to a naturally occurring Wnt-binding protein or a functional fragment thereof.

7. The method of claim 6 wherein the Wnt signalling antagonist comprises a Wnt-binding fragment of a naturally occurring Wnt-binding protein.

8. The method of claim 5 wherein the Wnt signalling antagonist comprises a protein at least 90% identical to a Wnt-binding fragment of an sFRP protein (soluble frizzled-related protein), WIF-1, or Cerberus.

9. The method of claim 8 wherein the Wnt signalling antagonist comprises a Wnt-binding fragment of an sFRP protein (soluble frizzled-related protein), WIF-1, or Cerberus.

10. The method of claim 5 wherein the Wnt signalling antagonist comprises a protein at least 90% identical to a Wnt-binding fragment of a Dickkopf protein.

11. The method of claim 10 wherein the Wnt signalling antagonist comprises a Wnt-binding fragment of a Dickkopf protein.

12. The method of claim 5 wherein the Wnt signalling antagonist comprises an antibody that binds to Wnt.

13. The method of claim 1 wherein the Wnt signalling antagonist comprises an agent that inhibits interaction between a canonical Wnt and Frizzled or LRP5/6.

14. The method of claim 13 wherein the Wnt signalling antagonist comprises an antibody that binds to Frizzled or LRP5/6.

15. The method of claim 1 wherein the subject is a human.

16. The method of claim 15 wherein the subject is a human diagnosed with scleroderma.

17. The method of claim 15 wherein the subject is a human who has been diagnosed as having decreased WIF1 expression in a skin biopsy.

18. A method of treating or preventing scleroderma, the method comprising:
administering, to a subject, an agent that comprises a functional fragment of WIF1, in an amount effective to treat or prevent scleroderma.

19. The method of claim 18 wherein the fragment is a Wnt-binding fragment of WIF1.

20. The method of claim 18 wherein the agent comprises full-length, mature WIF1.

21. A method of treating or preventing scleroderma, the method comprising:
administering, to a subject, an agent that increases activity or expression of WIF1 or an sFRP protein, in an amount effective to treat or prevent scleroderma.

22. A method of treating or preventing scleroderma, the method comprising:
administering, to a subject, a IGF binding agent, in an amount effective to treat or prevent scleroderma.

23. The method of claim 22 wherein the IGF binding agent binds to IGF-I or IGF-II.

24. The method of claim 22 wherein the IGF binding agent comprises IGF binding regions of a naturally occurring IGFBP.

25. The method of claim 22 wherein the IGFBP is IGFBP-3.

26. The method of claim 22 wherein the IGF binding agent comprises a full-length, mature IGFBP.

27. The method of claim 22 wherein the IGF binding agent comprises an antibody that binds to IGF-I or IGF-II.

28. A method of treating or preventing scleroderma, the method comprising:
administering, to a subject, an agent that (i) increases expression of a gene in Table 1, or (ii) increases activity of a gene product encoded by the gene, wherein the agent is administered in an amount effective to treat or prevent scleroderma.

29. The method of claim 28 wherein the agent is a nucleic acid that encodes a protein that comprises a functional fragment of the gene product.

30. The method of claim 29 wherein the nucleic acid is in a viral vector and delivered using viral particle.

31. The method of claim 29 wherein the agent comprises a functional fragment of the gene product.

32. The method of claim 29 wherein the agent comprises the gene product.

33. A method of treating or preventing scleroderma, the method comprising:
administering, to a subject, an agent that (i) decreases expression of a gene in Table 2, or (ii) decreases activity of a gene product encoded by the gene, wherein the agent is administered in an amount effective to treat or prevent scleroderma.

34. The method of claim 33 wherein the agent is a nucleic acid antagonist of gene expression.

35. The method of claim 34 wherein the nucleic acid antagonist is an RNAi.

36. The method of claim 34 wherein the agent is an antibody that binds to the gene product.

37. A method of evaluating a subject, the method comprising:
obtaining a sample from a subject; and
evaluating expression of WIF-1 in cells in the biopsy, wherein a decrease in WIF-1 expression relative to a reference is indicative of scleroderma or risk for scleroderma.

38. A method of evaluating a subject, the method comprising:
obtaining a sample from a subject; and
evaluating expression of a gene in Table 1 or 2 in cells in the biopsy, wherein an alteration in expression of the gene relative to a reference is indicative of scleroderma or risk for scleroderma.

39. The method of claim 38 wherein the gene is listed in Table 1 and a decrease in expression of the gene relative to a reference is indicative of scleroderma or risk for scleroderma.

40. The method of claim 38 wherein the gene is listed in Table 2 and an increase in expression of the gene relative to a reference is indicative of scleroderma or risk for scleroderma.

41. The method of claim 37 or 38 wherein the evaluating comprises a quantitative evaluation of expression levels.

42. The method of claim 37 or 38 wherein the evaluating comprises a qualitative evaluation of expression levels.

43. The method of claim 37 or 38 wherein the reference is a parameter obtained by evaluating a normal subject who does not have scleroderma.

44. The method of claim 37 or 38 wherein a plurality of genes are evaluated and the expression of each of the genes is compared to corresponding references.

45. The method of claim 37 or 38 wherein a plurality of genes are evaluated to obtain a profile of gene expression, and the profile is compared to a corresponding reference profile.

46. A method of evaluating a subject, the method comprising:
obtaining a sample from a subject; and
evaluating expression of a collagen in cells in the biopsy, wherein a increase in collagen expression relative to a reference is indicative of scleroderma or risk for scleroderma.

47. The method of claim 37, 38 or 46 wherein the sample comprises a skin biopsy.

48. The method of claim 37, 38 or 46 wherein the sample comprises a serum sample.

49. The method of claim 37, 38 or 46 further comprising, if the subject is indicated for scleroderma or risk for scleroderma, administering a therapy for scleroderma to the subject.

50. The method of claim 37, 38 or 46 further comprising preparing a report indicating a diagnosis of scleroderma or risk for scleroderma using results of the evaluating.

51. The method of claim 46 wherein the collagen is collagen XI.

52. A computer-readable database that comprises a plurality of records,
each record of the plurality comprising:
a) a first field that comprises information about skin pathology of a subject and;
b) a second field that comprises information about expression of a gene in Table 1 or 2 in cells from a skin biopsy obtained from the subject.

53. The database of claim 52 wherein each record of the plurality farther comprises a field that comprises information identifying the subject.

54. The database of claim 52 wherein each record of the plurality farther comprises fields, each additional field comprising information about expression of a gene in Table 1 or 2 such that each record includes information for a plurality of genes in Table 1 or 2.
